SEQUENCE LISTING (21 sequences)

SEQ ID NO: 1 - protein, 300 aa, Homo sapiens
MANCEFSPVSGDKPCCRLSRRAQLCLGVSILVLILVVVLAVVVPRWRQQWSGPGTTKRFPETVLARCVKYTEIHPEMRHVDCQSVWDAFKGAFISKHPCNITEEDYQPLMKLGTQTVPCNKILLWSRIKDLAHQFTQVQRDMFTLEDTLLGYLADDLTWCGEFNTSKINYQSCPDWRKDCSNNPVSVFWKTVSRRFAEAACDVVHVMLNGSRSKIFDKNSTFGSVEVHNLQPEKVQTLEAWVIHGGREDSRDLCQDPTIKELESIISKRNIQFSCKNIYRPDKFLQCVKNPEDSSCTSEI

SEQ ID NO: 2 - protein, 335 aa, Homo sapiens
MAGSPTCLTLIYILWQLTGSAASGPVKELVGSVGGAVTFPLKSKVKQVDSIVWTFNTTPLVTIQPEGGTIIVTQNRNRERVDFPDGGYSLKLSKLKKNDSGIYYVGIYSSSLQQPSTQEYVLHVYEHLSKPKVTMGLQSNKNGTCVTNLTCCMEHGEEDVIYTWKALGQAANESHNGSILPISWRWGESDMTFICVARNPVSRNFSSPILARKLCEGAADDPDSSMVLLCLLLVPLLLSLFVLGLFLWFLKRERQEEYIEEKKRVDICRETPNICPHSGENTEYDTIPHTNRTILKEDPANTVYSTVEIPKKMENPHSLLTMPDTPRLFAYENVI

SEQ ID NO: 3 - protein, 8 aa, synthetic construct
GYTFTSYW

SEQ ID NO: 4 - protein, 8 aa, synthetic construct
IYPGDGDT

SEQ ID NO: 5 - protein, 14 aa, synthetic construct
ARERTTGAPRYFDV

SEQ ID NO: 6 - protein, 6 aa, synthetic construct
ENIYSF

SEQ ID NO: 7 - skipped

SEQ ID NO: 8 - protein, 9 aa, synthetic construct
QHHYGIPLT

SEQ ID NO: 9 - protein, 121 aa, synthetic construct
QVQLQQSGAELARPGASVKLSCKASGYTFTSYWMQWVKQRPGQGLEWIGAIYPGDGDTRYTQKFKGKATLTADKSSSTAYMQLSNLTSEDSAVYYCARERTTGAPRYFDVWGAGTTVTVSS

SEQ ID NO: 10 - protein, 107 aa, synthetic construct
DIQMTQSPASLSASVGETVTITCRASENIYSFLAWYQQKQGKSPQLLVYNTKTLTEGVPSRFSGSGSGTQFSLKINNLQPEDFGSYYCQHHYGIPLTFGAGTKLELK

SEQ ID NO: 11 - protein, 243 aa, synthetic construct
DIQMTQSPASLSASVGETVTITCRASENIYSFLAWYQQKQGKSPQLLVYNTKTLTEGVPSRFSGSGSGTQFSLKINNLQPEDFGSYYCQHHYGIPLTFGAGTKLELKGGGGSGGGGSGGGGSQVQLQQSGAELARPGASVKLSCKASGYTFTSYWMQWVKQRPGQGLEWIGAIYPGDGDTRYTQKFKGKATLTADKSSSTAYMQLSNLTSEDSAVYYCARERTTGAPRYFDVWGAGTTVTVSS

SEQ ID NO: 12 - protein, 84 aa, synthetic construct
HFVPVFLPAKPTTTPAPRPPTPAPTIASQPLSLRPEACRPAAGGAVHTRGLDFACDIYIWAPLAGTCGVLLLSLVITLYCNHRN

SEQ ID NO: 13 - protein, 113 aa, synthetic construct
RVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 14 - protein, 442 aa, synthetic construct
DIQMTQSPASLSASVGETVTITCRASENIYSFLAWYQQKQGKSPQLLVYNTKTLTEGVPSRFSGSGSGTQFSLKINNLQPEDFGSYYCQHHYGIPLTFGAGTKLELKGGGGSGGGGSGGGGSQVQLQQSGAELARPGASVKLSCKASGYTFTSYWMQWVKQRPGQGLEWIGAIYPGDGDTRYTQKFKGKATLTADKSSSTAYMQLSNLTSEDSAVYYCARERTTGAPRYFDVWGAGTTVTVSSLEHFVPVFLPAKPTTTPAPRPPTPAPTIASQPLSLRPEACRPAAGGAVHTRGLDFACDIYIWAPLAGTCGVLLLSLVITLYCNHRNRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 15 - protein, 107 aa, synthetic construct
DIVMTQSQKSMSTSVGDRVSITCKASQDVITGVAWYQQKPGQSPKLLIYSASYRYTGVPDRFTGSGSGTDFTFTISNVQAEDLAVYYCQQHYSTPLTFGAGTKLELK

SEQ ID NO: 16 - protein, 120 aa, synthetic construct
SQVQLQQPGAELVRPGASVKLSCKASGYSFTTYWMNWVKQRPGQGLEWIGMIHPSDSETRLNQKFKDKATLTVDKSSSTAYMQLSSPTSEDSAVYYCARSTMIATRAMDYWGQGTSVTVS

SEQ ID NO: 17 - protein, 242 aa, synthetic construct
SQVQLQQPGAELVRPGASVKLSCKASGYSFTTYWMNWVKQRPGQGLEWIGMIHPSDSETRLNQKFKDKATLTVDKSSSTAYMQLSSPTSEDSAVYYCARSTMIATRAMDYWGQGTSVTVSGGGGSGGGGSGGGGSDIVMTQSQKSMSTSVGDRVSITCKASQDVITGVAWYQQKPGQSPKLLIYSASYRYTGVPDRFTGSGSGTDFTFTISNVQAEDLAVYYCQQHYSTPLTFGAGTKLELK

SEQ ID NO: 18 - protein, 42 aa, synthetic construct
KRGRKKLLYIFKQPFMRPVQTTQEEDGCSCRFPEEEEGGCEL

SEQ ID NO: 19 - protein, 333 aa, synthetic construct
SQVQLQQPGAELVRPGASVKLSCKASGYSFTTYWMNWVKQRPGQGLEWIGMIHPSDSETRLNQKFKDKATLTVDKSSSTAYMQLSSPTSEDSAVYYCARSTMIATRAMDYWGQGTSVTVSGGGGSGGGGSGGGGSDIVMTQSQKSMSTSVGDRVSITCKASQDVITGVAWYQQKPGQSPKLLIYSASYRYTGVPDRFTGSGSGTDFTFTISNVQAEDLAVYYCQQHYSTPLTFGAGTKLELKLEHFVPVFLPAKPTTTPAPRPPTPAPTIASQPLSLRPEACRPAAGGAVHTRGLDFACDIYIWAPLAGTCGVLLLSLVITLYCNHRNMHKRG

SEQ ID NO: 20 - protein, 867 aa, synthetic construct
SQVQLQQPGAELVRPGASVKLSCKASGYSFTTYWMNWVKQRPGQGLEWIGMIHPSDSETRLNQKFKDKATLTVDKSSSTAYMQLSSPTSEDSAVYYCARSTMIATRAMDYWGQGTSVTVSGGGGSGGGGSGGGGSDIVMTQSQKSMSTSVGDRVSITCKASQDVITGVAWYQQKPGQSPKLLIYSASYRYTGVPDRFTGSGSGTDFTFTISNVQAEDLAVYYCQQHYSTPLTFGAGTKLELKLEHFVPVFLPAKPTTTPAPRPPTPAPTIASQPLSLRPEACRPAAGGAVHTRGLDFACDIYIWAPLAGTCGVLLLSLVITLYCNHRNMHKRGRKKLLYIFKQPFMRPVQTTQEEDGCSCRFPEEEEGGCELGSGGSGATNFSLLKQAGDVEENPGPQRPGERVMALPVTALLLPLALLLHAARPDIQMTQSPASLSASVGETVTITCRASENIYSFLAWYQQKQGKSPQLLVYNTKTLTEGVPSRFSGSGSGTQFSLKINNLQPEDFGSYYCQHHYGIPLTFGAGTKLELKGGGGSGGGGSGGGGSQVQLQQSGAELARPGASVKLSCKASGYTFTSYWMQWVKQRPGQGLEWIGAIYPGDGDTRYTQKFKGKATLTADKSSSTAYMQLSNLTSEDSAVYYCARERTTGAPRYFDVWGAGTTVTVSSLEHFVPVFLPAKPTTTPAPRPPTPAPTIASQPLSLRPEACRPAAGGAVHTRGLDFACDIYIWAPLAGTCGVLLLSLVITLYCNHRNRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 21 - DNA, 2604 bp, synthetic construct
  source 1..2604: mol_type = other DNA
tcccaggtccaactgcagcagcctggggctgagctggtgaggcctggagcttcagtgaagctgtcctgcaaggcttcggggtactccttcaccacctactggatgaactgggtgaagcagaggcctggacaaggccttgagtggattggcatgattcatccttccgatagtgaaactaggttaaatcagaagttcaaggacaaggccacattgactgtagacaaatcctccagcacagcctacatgcaactcagcagcccgacatctgaggactctgcggtctattactgtgcaagatctactatgattgcgacgagggctatggactactggggtcaaggaacctcagtcaccgtctccggcggtggcggttctggtggcggtggctccggcggtggcggttctgacattgtgatgacccagtctcagaaatccatgtccacatcagtaggagacagggtcagcatcacctgcaaggccagtcaggatgttattactggtgtagcctggtatcaacagaaaccagggcaatctcctaaattactgatttactcggcatcctaccggtacactggagtccctgatcgcttcactggcagtggatctgggacggatttcactttcaccatcagcaatgtgcaggctgaagacctggcagtttattactgtcagcaacattatagtactcctctcactttcggtgctgggaccaagctggagctgaaactcgagcacttcgtgccggtcttcctgccagcgaagcccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaccacaggaacatgcataaacggggcagaaagaaactcctgtatatattcaaacaaccatttatgagaccagtacaaactactcaagaggaagatggctgtagctgccgatttccagaagaagaagaaggaggatgtgaactgggctccggaggatctggagcaacaaacttctcactactcaaacaagcaggtgacgtggaggagaatcccggcccccagcgtcctggggagcgcgtcatggccttaccagtgaccgccttgctcctgccgctggccttgctgctccacgccgccaggccggacatccagatgactcagtctccagcctccctatctgcatctgtgggggaaactgtcaccatcacatgtcgagcaagtgagaatatttacagttttttagcatggtatcagcagaaacagggaaaatctcctcagctcctggtctataatacaaaaaccttaacagaaggtgtgccatcaaggttcagtggcagtggatcaggcacacagttttctctgaagattaataacctgcagcctgaggattttgggagttattactgtcaacatcattatggtattccactcacgttcggtgctgggaccaagctggagctgaaaggcggtggcggttctggtggcggtggctccggcggtggcggttctcaggttcagctccagcagtctggggctgagctggcaagacctggggcttcagtgaagttgtcctgcaaggcttctggctacacctttacttcctactggatgcagtgggtaaaacagaggcctggacagggtctggaatggattggggctatttatcctggagatggtgatactaggtacactcagaagttcaagggcaaggccacattgactgcagataaatcctccagcacagcctacatgcaactcagcaacttgacatctgaggactctgcggtctattactgtgcaagagagaggactacgggagccccacggtacttcgatgtctggggcgcagggaccacggtcaccgtctcctcactcgagcacttcgtgccggtcttcctgccagcgaagcccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaccacaggaacagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

(19) United States
(12) Patent Application Publication
(10) Pub. No.: US 2026/0345680 A1
(43) Pub. Date: Nov. 5, 2026

(54) NOVEL DUAL SPLIT CAR-T CELLS FOR THE TREATMENT OF CD38-POSITIVE HEMATOLOGICAL MALIGNANCIES

(71) Applicants: INSTITUT NATIONAL DE LA SANTÉ ET DE LA RECHERCHE MÉDICALE, PARIS (FR); ASSISTANCE PUBLIQUE-HÔPITAUX DE PARIS (APHP), PARIS (FR); UNIVERSITÉ PARIS CITÉ, PARIS (FR); IFM SAS, PARIS (FR)

(72) Inventors: Jean-Christophe BORIES, PARIS (FR); Bertrand ARNULF, PARIS (FR); Jean-Paul FERMAND, PARIS (FR); Nathalie RODERS, PARIS (FR)

(21) Appl. No.: 19/106,041

(22) PCT Filed: Sep. 5, 2023
(86) PCT No.: PCT/EP2023/074271

§ 371 Date: Feb. 24, 2025

(30) Foreign Application Priority Data
Sep. 6, 2022 (EP) 22306319.9

(51) Int. Cl. A61K 35/17; A61K 9/00; A61K 40/11; A61K 40/15; A61K 40/31; A61K 40/42; A61P 35/00; C07K 14/705; C07K 16/00; C12N 5/0735; C12N 5/074; C12N 5/0783

(52) U.S. Cl. CPC A61K 35/17; A61K 9/0019; A61K 40/11; A61K 40/15; A61K 40/31; A61K 40/4222; A61P 35/00; C07K 14/70503; C07K 16/00; C12N 5/0606; C12N 5/0607; C12N 5/0638; C12N 5/0646; A61K 2239/21; A61K 2239/22; A61K 2239/28; A61K 2239/48; C07K 2317/565; C07K 2317/622; C07K 2319/02; C07K 2319/03; C12N 2510/00

(57) ABSTRACT

Chimeric antigen receptor (CAR) T-cell therapy for multiple myeloma (MM) targeting B-cell maturation antigen (BC-MA) induces high overall response rates. However, relapse occurs frequently and novel strategies for CAR-T cells targeting MM cells are needed. CAR-T cells targeting CD38 represent a potential alternative, but expression of CD38 on activated T cells and other hematopoietic cells raises concerns about the efficacy and safety of such therapy. Here, the inventors developed DCAR, a double CAR system targeting CD38 and SLAMF7 (CS1) through split activation and co-stimulation receptors, respectively. Furthermore, the inventors show that CRISPR/Cas9 inactivation of the CD38 gene enhances the anti-MM activity of DCAR-T in vitro. Edited DCAR-T developed strong responses specifically against MM cells expressing both the CD38 and the CS1 antigens in vitro and in vivo. Importantly, the inventors bring evidence that, unlike anti-CD38 CAR-T, which elicited a severe immune reaction against hematopoietic cells in a humanized mouse model, DCAR-T showed no sign of toxicity. Accordingly, the present invention relates to novel dual split CAR-T cells and uses thereof for the treatment of CD38-positive hematological malignancies.

### FIELD OF THE INVENTION

The present invention is in the field of medicine, in particular oncology and immunology.

### BACKGROUND OF THE INVENTION

Multiple myeloma (MM) is an hematological malignancy characterized by the clonal proliferation of tumour plasma cells in the bone marrow (Morgan et al., 2012). Despite significant therapeutic advances through the development of proteasome inhibitors (PI), immunomodulators (IMIDs), and hematopoietic stem cell transplantation, many MM patients relapse and develop refractory/resistant (R/R) diseases (Dimopoulos et al., 2022). New strategies, based on the combination of PI, IMIDs and monoclonal antibodies (mAb) targeting the CD38 antigen, have significantly improve the prognosis (Dimopoulos et al., 2016). Nevertheless, most patients still relapse and MM remains an incurable disease.

Recent clinical studies have shown that T cells expressing Chimeric Antigen Receptors (CAR-T) represents an efficient therapeutic approach in several B cell malignancies (Boyiadzis et al., 2018). Chimeric Antigen Receptors are hybrid molecules associating an extracellular portion, involved in antigen-recognition, with a transmembrane region fused with signalling domains including (in most cases) the CD3z and CD28 and/or the CD137 receptor, also referred to as 4-1 in (Sadelain et al., 2013). In this setting, the CD3z provides an activation signal and the 4-1 in region provides a co-stimulation signal, both of which are mandatory to mimic the physiological T cell mechanisms required for cytotoxicity, differentiation and persistence of T cells in vivo. Such CAR T cells targeting the B cell maturation antigen (BCMA) have brought up to 80% response rates in MM patients (depending on studies), however, the median overall survival remains below 25 months (Munshi et al., 2021). Although BCMA is detected on most post germinal centre B lineage cells, expression on plasma cells can vary through several mechanisms, including antigen loss or expression shedding, potentially leading to variable responses (Da Via et al., 2021; Laurent et al., 2015). Thus, developing immunotherapies for additional targets may mitigate antigen loss and effectively treat patients with low or variable BCMA expression.

Several antigens expressed at the surface of tumor plasma cells, such as GRPCD5, SLAMF7 (CS1) or CD38 are interesting targets and CAR-T directed against these receptors are been evaluated. CD38 is a glycoprotein with cyclic ADP ribose hydrolase activities, which is expressed on tumor plasma cells (and normal plasma cells) as well as on other lymphoid and myeloid cell populations. It was originally identified as a T and B lymphocyte activation marker and was later shown to be expressed on multiple haematopoietic cells (HSC), including subsets of haematopoietic stem cells, NK cells and monocytes. Recently, anti-CD38 CAR T cells have shown good anti-MM activities in pre-clinical models (Drent et al. Mol Ther. 2017). However, given the pattern of CD38 expression, such anti-CD38 CAR-T cell therapy might trigger side effects such as fratricide killing of activated CAR-T cells, as well as toxicity against myeloid cells, HSC or non-hematopoietic CD38 expressing cells (ie, endothelial cells). Thus, while CD38 represents a validated target for immune therapy in MM, improvements are needed to deliver safer and more efficient anti-CD38 CAR-T.

It has been suggested that dual antigen targeting by CAR T-cells can be exploited to establish MM-specificity of CAR T-cell therapy by the application of split dual CAR T-cell strategy (Kloss et al., 2013; Lanitis et al., 2013).

### SUMMARY OF THE INVENTION

The present invention is defined by the claims. In particular, the present invention relates to novel dual split CAR-T cells and uses thereof for the treatment of CD38-positive hematological malignancies.

### DETAILED DESCRIPTION OF THE INVENTION

Chimeric antigen receptor (CAR) T-cell therapy for multiple myeloma (MM) targeting B-cell maturation antigen (BCMA) induces high overall response rates. However, relapse occurs frequently and novel strategies for CAR-T cells targeting MM cells are needed. CAR-T cells targeting CD38 represent a potential alternative, but expression of CD38 on activated T cells and other hematopoietic cells raises concerns about the efficacy and safety of such therapy.

Here, the inventors developed DCAR, a double CAR system targeting CD38 and SLAMF7 (CS1) through split activation and co-stimulation receptors, respectively. Furthermore, the inventors show that CRISPR/Cas9 inactivation of the CD38 gene enhances the anti-MM activity of DCAR-T in vitro. Edited DCAR-T developed strong responses specifically against MM cells expressing both the CD38 and the CS1 antigens in vitro and in vivo. Importantly, the inventors bring evidence that, unlike anti-CD38 CAR-T, which elicited a severe immune reaction against hematopoietic cells in a humanized mouse model, DCAR-T showed no sign of toxicity. Thus, DCAR-T cells provides a safe and efficient alternative to treat MM patients.

### MAIN DEFINITIONS

As used herein, the terms “polypeptide”, “peptide”, and “protein” are used interchangeably herein to refer to polymers of amino acids of any length. The terms also encompass an amino acid polymer that has been modified; for example, disulfide bond formation, glycosylation, lipidation, phosphorylation, or conjugation with a labeling component. Polypeptides when discussed in the context of gene therapy refer to the respective intact polypeptide, or any fragment or genetically engineered derivative thereof, which retains the desired biochemical function of the intact protein.

As used herein, the term “polynucleotide” refers to a polymeric form of nucleotides of any length, including deoxyribonucleotides or ribonucleotides, or analogs thereof. A polynucleotide may comprise modified nucleotides, such as methylated nucleotides and nucleotide analogs, and may be interrupted by non-nucleotide components. If present, modifications to the nucleotide structure may be imparted before or after assembly of the polymer. The term polynucleotide, as used herein, refers interchangeably to double- and single-stranded molecules. Unless otherwise specified or required, any embodiment of the invention described herein that is a polynucleotide encompasses both the double-stranded form and each of two complementary single-stranded forms known or predicted to make up the double-stranded form.

As used herein, the expression “derived from” refers to a process whereby a first component (e.g., a first polypeptide), or information from that first component, is used to isolate, derive or make a different second component (e.g., a second polypeptide that is different from the first one).

As used herein, the “percent identity” between the two sequences is a function of the number of identical positions shared by the sequences (i.e., % identity=number of identical positions/total number of positions×100), taking into account the number of gaps, and the length of each gap, which need to be introduced for optimal alignment of the two sequences. The comparison of sequences and determination of percent identity between two sequences can be accomplished using a mathematical algorithm, as described below. The percent identity between two amino acid sequences can be determined using the Needleman and Wunsch algorithm (Needleman, Saul B. & Wunsch, Christian D. (1970). “A general method applicable to the search for similarities in the amino acid sequence of two proteins”. Journal of Molecular Biology. 48 (3): 443-53.). The percent identity between two nucleotide or amino acid sequences may also be determined using for example algorithms such as EMBOSS Needle (pair wise alignment; available at www.ebi.ac.uk). For example, EMBOSS Needle may be used with a BLOSUM62 matrix, a “gap open penalty” of 10, a “gap extend penalty” of 0.5, a false “end gap penalty”, an “end gap open penalty” of 10 and an “end gap extend penalty” of 0.5. In general, the “percent identity” is a function of the number of matching positions divided by the number of positions compared and multiplied by 100. For instance, if 6 out of 10 sequence positions are identical between the two compared sequences after alignment, then the identity is 60%. The % identity is typically determined over the whole length of the query sequence on which the analysis is performed. Two molecules having the same primary amino acid sequence or nucleic acid sequence are identical irrespective of any chemical and/or biological modification. According to the invention, a first amino acid sequence having at least 90% of identity with a second amino acid sequence means that the first sequence has 90; 91; 92; 93; 94; 95; 96; 97; 98; 99 or 100% of identity with the second amino acid sequence.

As used herein, the term “engineered” refers to an aspect of having been manipulated and altered by the hand of man. In particular, the term “engineered cell” refers to a cell that has been subjected to a manipulation, so that its genetic, epigenetic, and/or phenotypic identity is altered relative to an appropriate reference cell such as otherwise identical cell that has not been so manipulated. In some embodiments, the manipulation is or comprises a genetic manipulation. In some embodiments, a genetic manipulation is or comprises one or more of (i) introduction of a nucleic acid not present in the cell prior to the manipulation (i.e., of a heterologous nucleic acid); (ii) removal of a nucleic acid, or portion thereof, present in the cell prior to the manipulation; and/or (iii) alteration (e.g., by sequence substitution) of a nucleic acid, or portion thereof, present in the cell prior to the manipulation. In some embodiments, an engineered cell is one that has been manipulated so that it contains and/or expresses a particular agent of interest (e.g., a protein, a nucleic acid, and/or a particular form thereof) in an altered amount and/or according to altered timing relative to such an appropriate reference cell. Those of ordinary skill in the art will appreciate that reference to an “engineered cell” herein may, in some embodiments, encompass both the particular cell to which the manipulation was applied and also any progeny of such cell.

As used herein, the term “expression” of a nucleic acid sequence refers to one or more of the following events: (1) production of an RNA template from a DNA sequence (e.g., by transcription); (2) processing of an RNA transcript (e.g., by splicing, editing, 5′ cap formation, and/or 3′ end formation); (3) translation of an RNA into a polypeptide or protein; and/or (4) post-translational modification of a polypeptide or protein.

As used herein, the term “encoding” refers to the inherent property of specific sequences of nucleotides in a polynucleotide, such as, for example, a gene, a cDNA, or a mRNA, to serve as templates for synthesis of other polymers and macromolecules in biological processes having either a defined sequence of nucleotides (e.g., rRNA, tRNA and mRNA) or a defined sequence of amino acids and the biological properties resulting therefrom. Thus, a gene, cDNA, or RNA, encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA. Unless otherwise specified, a “polynucleotide sequence encoding an amino acid sequence” includes all nucleotide sequences that are degenerate versions of each other and that encode the same amino acid sequence.

As used herein, the term “CD38” has its general meaning in the art and refers to the ADP-ribosyl cyclase/cyclic ADP-ribose hydrolase 1. An exemplary amino acid sequence for CD38 is represented by SEQ ID NO:1. The extracellular domain of CD38 ranges from the amino acid residue at position 43 to the amino acid residue at position 300 in SEQ ID NO:1.

>sp|P28907|CD38_HUMAN ADP-ribosyl cyclase/cyclic ADP-
ribose hydrolase 1 OS = Homo sapiens
OX = 9606 GN = CD38 PE = 1 SV = 2
SEQ ID NO: 1
MANCEFSPVSGDKPCCRLSRRAQLCLGVSILVLILVVVLAVVVPRWRQQWSGPGTTKREP
ETVLARCVKYTEIHPEMRHVDCQSVWDAFKGAFISKHPCNITEEDYQPLMKLGTQTVPCN
KILLWSRIKDLAHQFTQVQRDMFTLEDTLLGYLADDLTWCGEFNTSKINYQSCPDWRKDC
SNNPVSVFWKTVSRRFAEAACDVVHVMLNGSRSKIFDKNSTFGSVEVHNLQPEKVQTLEA
WVIHGGREDSRDLCQDPTIKELESIISKRNIQFSCKNIYRPDKFLQCVKNPEDSSCTSEI

As used herein, the term “SLAMF7” has its general meaning in the art and refers to the SLAM family member 7 protein. The term is also known as CD2 subset 1, CD2-like receptor-activating cytotoxic cells (CRACC), Membrane protein FOAP-12, Novel Ly9, Protein 19A, and CD319.

An exemplary amino acid sequence for SLAMF7 is represented by SEQ ID NO:2. The extracellular domain of SLAMF7 ranges from the amino acid residue at position 23 to the amino acid residue at position 226 in SEQ ID NO:2.

>sp|Q9NQ25|SLAF7 HUMAN SLAM family member 7
OS = Homo sapiens OX = 9606 GN = SLAMF7 PE = 1 SV = 1
SEQ ID NO: 2
MAGSPTCLTLIYILWQLTGSAASGPVKELVGSVGGAVTFPLKSKVKQVDSIVWTENTTPL
VTIQPEGGTIIVTQNRNRERVDFPDGGYSLKLSKLKKNDSGIYYVGIYSSSLQQPSTQEY
VLHVYEHLSKPKVTMGLQSNKNGTCVTNLTCCMEHGEEDVIYTWKALGQAANESHNGSIL
PISWRWGESDMTFICVARNPVSRNESSPILARKLCEGAADDPDSSMVLLCLLLVPLLLSL
FVLGLFLWFLKRERQEEYIEEKKRVDICRETPNICPHSGENTEYDTIPHTNRTILKEDPA
NTVYSTVEIPKKMENPHSLLTMPDTPRLFAYENVI

As used herein, the term “subject”, “host”, “individual” or “patient” refers to a mammal, preferably a human being, male or female at any age that is in-need of a therapy.

As used herein, the term “cell” refers to any eukaryotic cell. In some embodiments the cells are selected from the group consisting of multipotent hematopoietic stem cells derived from bone marrow, peripheral blood, or umbilical cord blood; or pluripotent (i.e. embryonic stem cells (ES) or induced pluripotent stem cells (iPS)) or multipotent stem cell-derived differentiated cells of different cell lineages.

A used herein, the term “host cell” or “recipient cell” refers to a cell that was genetically engineered, i.e. harboring an exogenous nucleotide sequence, preferably stably integrated, in its genome.

As used herein, the term “immune cell” refers to a cell that functions in an immune response or a progenitor, or progeny thereof.

As used herein, the term “population” refers to a population of cells, wherein the majority (e.g., at least about 50%, preferably at least about 60%, more preferably at least about 70%, and even more preferably at least about 80%) of the total number of cells have the specified characteristics of the cells of interest and express the markers of interest (e.g. a population of human CAR-host immune cells comprises at least about 50%, preferably at least about 60%, more preferably at least about 70%, and even more preferably at least about 80% of cells which have the highly suppressive functions and which express the particular markers of interest).

As used herein, the term “T cell” has its general meaning in the art and represent an important component of the immune system that plays a central role in cell-mediated immunity. T cells are known as conventional lymphocytes as they recognize the antigen with their TCR (T cell receptor for the antigen) with presentation or restriction by molecules of the complex major histocompatibility. There are several subsets of T cells each having a distinct function such as CD8+ T cells, CD4+ T cells, and gamma delta T cells. Cytotoxic T cells (CTL or killer T cells) are a subset of T lymphocytes capable of inducing the death of infected somatic or tumor cells.

As used herein, the term “chimeric antigen receptor” or “CAR” has its general meaning in the art and refers to an artificially constructed hybrid protein or polypeptide containing the antigen binding domains of an antibody (e.g., scFv) linked to T-cell signalling domains.

Characteristics of CARs include their ability to redirect T-cell specificity and reactivity toward a selected target in a non-MHC-restricted manner, exploiting the antigen-binding properties of monoclonal antibodies. Moreover, when expressed in T-cells, CARs advantageously do not dimerize with endogenous T cell receptor (TCR) alpha and beta chains. The chimeric antigen receptor of the present invention typically comprises an extracellular hinge domain, a transmembrane domain, and an intracellular T cell signalling domain.

As used herein, the term “chimeric co-stimulatory receptor” or “CCR” refers to a specific type of chimeric antigen receptor (CAR) that mediates costimulation independently of activation. When expressed on host immune cells in combination with a CAR, the CCR is targeted to a second antigen.

As used herein, the term “CAR-T cell” refers to a T lymphocyte that has been genetically engineered to express a CAR. The T lymphocytes that are genetically modified may be “derived” or “obtained” from the patient who will receive the treatment using the genetically modified T cells or they may be “derived” or “obtained” from a different patient.

As used herein, the term “DCAR-T cell” refers to a T lymphocyte that has been genetically engineered to express both a CAR and a CCR.

As used herein, the term “cytotoxicity” refers to ability of the host immune cell of the present invention to lyse target cells. Such cytotoxicity can be measured using standard techniques, e.g., by radioactively labeling the target cells. Typically said activity is measured according to the methods described in the EXAMPLE. The expression “negligible cytotoxicity” means that the cytotoxicity is reduced by 50, 60, 70, 80, 85, 90, 95, 99 or 100%.

As used herein, the term “antigen” has its general meaning in the art and generally refers to a substance or fragment thereof that is recognized and selectively bound by an antibody or by a T cell antigen receptor, resulting in induction of an immune response. Antigens according to the invention are typically, although not exclusively, peptides and proteins. Antigens may be natural or synthetic and generally induce an immune response that is specific for that antigen.

As used herein the term “antibody” and “immunoglobulin” have the same meaning, and will be used equally in the present invention. The term “antibody” as used herein refers to immunoglobulin molecules and immunologically active portions of immunoglobulin molecules, i.e., molecules that contain an antigen binding site that immunospecifically binds an antigen. As such, the term antibody encompasses not only whole antibody molecules, but also antibody fragments as well as variants (including derivatives) of antibodies and antibody fragments. In natural antibodies, two heavy chains are linked to each other by disulfide bonds and each heavy chain is linked to a light chain by a disulfide bond. There are two types of light chain, lambda (l) and kappa (k). There are five main heavy chain classes (or isotypes) which determine the functional activity of an antibody molecule: IgM, IgD, IgG, IgA and IgE. Each chain contains distinct sequence domains. The light chain includes two domains, a variable domain (VL) and a constant domain (CL). The heavy chain includes three (α, δ, γ) to five (μ, ε) domains, a variable domain (VH) and three to four constant domains (CH1, CH2, CH3 and CH4 collectively referred to as CH). The variable regions of both light (VL) and heavy (VH) chains determine binding recognition and specificity to the antigen. The constant region domains of the light (CL) and heavy (CH) chains confer important biological properties such as antibody chain association, secretion, trans-placental mobility, complement binding, and binding to Fc receptors (FcR). The Fv fragment is the N-terminal part of the Fab fragment of an immunoglobulin and consists of the variable portions of one light chain and one heavy chain. The specificity of the antibody resides in the structural complementarity between the antibody combining site and the antigenic determinant. Antibody combining sites are made up of residues that are primarily from the hypervariable or complementarity determining regions (CDRs). Occasionally, residues from nonhypervariable or framework regions (FR) can participate to the antibody binding site or influence the overall domain structure and hence the combining site. CDRs refer to amino acid sequences which together define the binding affinity and specificity of the natural Fv region of a native immunoglobulin binding site. The light and heavy chains of an immunoglobulin each have three CDRs, designated L-CDR1, L-CDR2, L-CDR3 and H-CDR1, H-CDR2, H-CDR3, respectively. An antigen-binding site, therefore, typically includes six CDRs, comprising the CDR set from each of a heavy and a light chain V region. Framework Regions (FRs) refer to amino acid sequences interposed between CDRs. The residues in antibody variable domains are conventionally numbered according to a system devised by Kabat et al. This system is set forth in Kabat et al., 1987, in Sequences of Proteins of Immunological Interest, US Department of Health and Human Services, NIH, USA (hereafter “Kabat et al.”). This numbering system is used in the present specification. The Kabat residue designations do not always correspond directly with the linear numbering of the amino acid residues in SEQ ID sequences. The actual linear amino acid sequence may contain fewer or additional amino acids than in the strict Kabat numbering corresponding to a shortening of, or insertion into, a structural component, whether framework or complementarity determining region (CDR), of the basic variable domain structure. The correct Kabat numbering of residues may be determined for a given antibody by alignment of residues of homology in the sequence of the antibody with a “standard” Kabat numbered sequence. The CDRs of the heavy chain variable domain are located at residues 31-35B (H-CDR1), residues 50-65 (H-CDR2) and residues 95-102 (H-CDR3) according to the Kabat numbering system. The CDRs of the light chain variable domain are located at residues 24-34 (L-CDR1), residues 50-56 (L-CDR2) and residues 89-97 (L-CDR3) according to the Kabat numbering system.

As used herein, the terms “monoclonal antibody”, “monoclonal Ab”, “monoclonal antibody composition”, “mAb”, or the like, as used herein refer to a preparation of antibody molecules of single molecular composition. A monoclonal antibody is obtained from a population of substantially homogeneous antibodies, i.e., the individual antibodies comprised in the population are identical except for possible naturally occurring mutations that may be present in minor amounts.

As used herein the term “human antibody” as used herein, is intended to include antibodies having variable and constant regions derived from human immunoglobulin sequences. The human antibodies of the present invention may include amino acid residues not encoded by human immunoglobulin sequences (e.g., mutations introduced by random or site-specific mutagenesis in vitro or by somatic mutation in vivo). However, the term “human antibody”, as used herein, is not intended to include antibodies in which CDR sequences derived from the germline of another mammalian species, such as a mouse, have been grafted onto human framework sequences.

As used herein, the term “chimeric antibody” refers to an antibody which comprises a VH domain and a VL domain of a non-human antibody, and a CH domain and a CL domain of a human antibody. In some embodiments, a “chimeric antibody” is an antibody molecule in which (a) the constant region (i.e., the heavy and/or light chain), or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, hormone, growth factor, drug, etc.; or (b) the variable region, or a portion thereof, is altered, replaced or exchanged with a variable region having a different or altered antigen specificity. Chimeric antibodies also include primatized and in particular humanized antibodies. Furthermore, chimeric antibodies may comprise residues that are not found in the recipient antibody or in the donor antibody. These modifications are made to further refine antibody performance. For further details, see Jones et al., Nature 321:522-525 (1986); Riechmann et al., Nature 332:323-329 (1988); and Presta, Curr. Op. Struct. Biol. 2:593-596 (1992). (see U.S. Pat. No. 4,816,567; and Morrison et al., Proc. Natl. Acad. Sci. USA, 81:6851-6855 (1984)).

As used hereon, the term “humanized antibody” refers to an antibody having variable region framework and constant regions from a human antibody but retains the CDRs of a previous non-human antibody. In some embodiments, a humanized antibody contains minimal sequence derived from non-human immunoglobulin. For the most part, humanized antibodies and antibody fragments thereof may be human immunoglobulins (recipient antibody or antibody fragment) in which residues from a complementary-determining region (CDR) of the recipient are replaced by residues from a CDR of a non-human species (donor antibody) such as mouse, rat or rabbit having the desired specificity, affinity, and capacity. In some instances, Fv framework region (FR) residues of the human immunoglobulin are replaced by corresponding non-human residues. Furthermore, a humanized antibody/antibody fragment can comprise residues which are found neither in the recipient antibody nor in the imported CDR or framework sequences. Such antibodies are designed to maintain the binding specificity of the non-human antibody from which the binding regions are derived, but to avoid an immune reaction against the non-human antibody. These modifications can further refine and optimize antibody or antibody fragment performance. In general, the humanized antibody or antibody fragment thereof will comprise substantially all of at least one, and typically two, variable domains, in which all or substantially all of the CDR regions correspond to those of a non-human immunoglobulin and all or a significant portion of the FR regions are those of a human immunoglobulin sequence. The humanized antibody or antibody fragment can also comprise at least a portion of an immunoglobulin constant region (Fc), typically that of a human immunoglobulin. For further details, see Jones et al., Nature, 321: 522-525, 1986; Reichmann et al., Nature, 332: 323-329, 1988; Presta, Curr. Op. Struct. Biol., 2: 593-596, 1992.

As used herein, the term “antibody fragment” refers to at least one portion of an intact antibody, preferably the antigen binding region or variable region of the intact antibody, that retains the ability to specifically interact with (e.g., by binding, steric hindrance, stabilizing/destabilizing, spatial distribution) an epitope of an antigen. “Fragments” comprise a portion of the intact antibody, generally the antigen binding site or variable region. Examples of antibody fragments include Fab, Fab′, Fab′-SH, F(ab′)2, and Fv fragments; diabodies; any antibody fragment that is a polypeptide having a primary structure consisting of one uninterrupted sequence of contiguous amino acid residues (referred to herein as a “single-chain antibody fragment” or “single chain polypeptide”), including without limitation (1) single-chain Fv molecules (2) single chain polypeptides containing only one light chain variable domain, or a fragment thereof that contains the three CDRs of the light chain variable domain, without an associated heavy chain moiety and (3) single chain polypeptides containing only one heavy chain variable region, or a fragment thereof containing the three CDRs of the heavy chain variable region, without an associated light chain moiety; and multispecific antibodies formed from antibody fragments. Fragments of the present antibodies can be obtained using standard methods.

As used herein, the term “scFv” refers to a fusion protein comprising at least one antibody fragment comprising a variable region of a light chain and at least one antibody fragment comprising a variable region of a heavy chain, wherein the light and heavy chain variable regions are contiguously linked, e.g., via a synthetic linker, e.g., a short flexible polypeptide linker, and capable of being expressed as a single chain polypeptide, and wherein the scFv retains the specificity of the intact antibody from which it is derived. Unless specified, as used herein an scFv may have the VL and VH variable regions in either order, e.g., with respect to the N-terminal and C-terminal ends of the polypeptide, the scFv may comprise VL-linker-VH or may comprise VH-linker-VL.

As used herein, the term “specificity” refers to the ability of an antibody to detectably bind target molecule (e.g. an epitope presented on an antigen) while having relatively little detectable reactivity with other target molecules. Specificity can be relatively determined by binding or competitive binding assays, using, e.g., Biacore instruments, as described elsewhere herein.

Specificity can be exhibited by, e.g., an about 10:1, about 20:1, about 50:1, about 100:1, 10.000:1 or greater ratio of affinity/avidity in binding to the specific antigen versus nonspecific binding to other irrelevant molecules.

The term “affinity”, as used herein, means the strength of the binding of an antibody to a target molecule (e.g. an epitope). The affinity of a binding protein is given by the dissociation constant Kd. For an antibody said Kd is defined as [Ab]×[Ag]/[Ab−Ag], where [Ab−Ag] is the molar concentration of the antibody-antigen complex, [Ab] is the molar concentration of the unbound antibody and [Ag] is the molar concentration of the unbound antigen. The affinity constant Ka is defined by 1/Kd. Preferred methods for determining the affinity of a binding protein can be found in Harlow, et al., Antibodies: A Laboratory Manual, Cold Spring Harbor Laboratory Press, Cold Spring Harbor, N.Y., 1988), Coligan et al., eds., Current Protocols in Immunology, Greene Publishing Assoc. and Wiley Interscience, N.Y., (1992, 1993), and Muller, Meth. Enzymol. 92:589-601 (1983), which references are entirely incorporated herein by reference. One preferred and standard method well known in the art for determining the affinity of binding protein is the use of Biacore instruments.

The term “binding” as used herein refers to a direct association between two molecules, due to, for example, covalent, electrostatic, hydrophobic, and ionic and/or hydrogen-bond interactions, including interactions such as salt bridges and water bridges. In particular, as used herein, the term “binding” in the context of the binding of an antibody to a predetermined target molecule (e.g. an antigen or epitope) typically is a binding with an affinity corresponding to a K_{D} of about 10⁻⁷ M or less, such as about 10⁻⁸ M or less, such as about 10⁻⁹ M or less, about 10⁻¹⁰ M or less, or about 10⁻¹¹ M or even less.

As used herein, the term “cancer” has its general meaning in the art and includes, but is not limited to, solid tumors and blood-borne tumors.

As uses herein, the term “CD38-positive hematological malignancy” refers to a hematological malignancy characterized by the presence of tumor cells expressing CD38 including leukemias, lymphomas and myeloma. Examples of such CD38-positive hematological malignancies include precursor B-cell lymphoblastic leukemia/lymphoma and B-cell non-Hodgkin's lymphoma; acute promyelocytic leukemia, acute lymphoblastic leukemia and mature B-cell neoplasms, such as B-cell chronic lymphocytic leukemia (CLL)/small lymphocytic lymphoma (SLL), B-cell acute lymphocytic leukemia, B-cell prolymphocytic leukemia, lymphoplasmacytic lymphoma, mantle cell lymphoma (MCL), follicular lymphoma (FL), including low-grade, intermediate-grade and high-grade FL, cutaneous follicle center lymphoma, marginal zone B-cell lymphoma (MALT type, nodal and splenic type), hairy cell leukemia, diffuse large B-cell lymphoma (DLBCL), Burkitt's lymphoma (BL), plasmacytoma, multiple myeloma, plasma cell leukemia, post-transplant lymphoproliferative disorder, Waldenstrom's macroglobulinemia, plasma cell leukemias and anaplastic large-cell lymphoma (ALCL).

As used herein, the term “treatment” or “treat” refer to both prophylactic or preventive treatment as well as curative or disease modifying treatment, including treatment of patient at risk of contracting the disease or suspected to have contracted the disease as well as patients who are ill or have been diagnosed as suffering from a disease or medical condition, and includes suppression of clinical relapse. The treatment may be administered to a patient having a medical disorder or who ultimately may acquire the disorder, in order to prevent, cure, delay the onset of, reduce the severity of, or ameliorate one or more symptoms of a disorder or recurring disorder, or in order to prolong the survival of a patient beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a patient during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a patient during treatment of an illness, e.g., to keep the patient in remission for long periods of time (months or years). A maintenance regimen may employ continuous therapy (e.g., administering a drug at a regular interval, e.g., weekly, monthly, yearly, etc.) or intermittent therapy (e.g., interrupted treatment, intermittent treatment, treatment at relapse, or treatment upon achievement of a particular predetermined criteria [e.g., pain, disease manifestation, etc.]).

As used herein, the term “therapeutically effective amount” refers to an amount effective, at dosages and for periods of time necessary, to achieve a desired therapeutic result. A therapeutically effective amount of the active agent may vary according to factors such as the disease state, age, sex, and weight of the individual, and the ability of the active agent to elicit a desired response in the individual. A therapeutically effective amount is also one in which any toxic or detrimental effects of the antibody or antibody portion are outweighed by the therapeutically beneficial effects. The efficient dosages and dosage regimens for the active agent depend on the disease or condition to be treated and may be determined by the persons skilled in the art. A physician having ordinary skill in the art may readily determine and prescribe the effective amount of the pharmaceutical composition required. For example, the physician could start doses of active agent employed in the pharmaceutical composition at levels lower than that required achieving the desired therapeutic effect and gradually increasing the dosage until the desired effect is achieved. In general, a suitable dose of a composition of the present invention will be that amount of the compound, which is the lowest dose effective to produce a therapeutic effect according to a particular dosage regimen. Such an effective dose will generally depend upon the factors described above. For example, a therapeutically effective amount for therapeutic use may be measured by its ability to stabilize the progression of disease.

Typically, the ability of a compound to inhibit cancer may, for example, be evaluated in an animal model system predictive of efficacy in human tumors. A therapeutically effective amount of a therapeutic compound may decrease tumor size, or otherwise ameliorate symptoms in a patient. One of ordinary skill in the art would be able to determine such amounts based on such factors as the patient's size, the severity of the patient's symptoms, and the particular composition or route of administration selected. An exemplary, non-limiting range for a therapeutically effective amount of a inhibitor of the present invention is about 0.1-100 mg/kg, such as about 0.1-50 mg/kg, for example about 0.1-20 mg/kg, such as about 0.1-10 mg/kg, for instance about 0.5, about such as 0.3, about 1, about 3 mg/kg, about 5 mg/kg or about 8 mg/kg.

An exemplary, non-limiting range for a therapeutically effective amount of a inhibitor of the present invention is 0.02-100 mg/kg, such as about 0.02-30 mg/kg, such as about 0.05-10 mg/kg or 0.1-3 mg/kg, for example about 0.5-2 mg/kg. Administration may e.g. be intravenous, intramuscular, intraperitoneal, or subcutaneous, and for instance administered proximal to the site of the target. Dosage regimens in the above methods of treatment and uses are adjusted to provide the optimum desired response (e.g., a therapeutic response). For example, a single bolus may be administered, several divided doses may be administered over time or the dose may be proportionally reduced or increased as indicated by the exigencies of the therapeutic situation.

In some embodiments, the efficacy of the treatment is monitored during the therapy, e.g. at predefined points in time. In some embodiments, the efficacy may be monitored by visualization of the disease area, or by other diagnostic methods described further herein, e.g. by performing one or more PET-CT scans, for example using a labeled inhibitor of the present invention, fragment or mini-antibody derived from the inhibitor of the present invention. If desired, an effective daily dose of a pharmaceutical composition may be administered as two, three, four, five, six or more sub-doses administered separately at appropriate intervals throughout the day, optionally, in unit dosage forms. In some embodiments, the human monoclonal antibodies of the present invention are administered by slow continuous infusion over a long period, such as more than 24 hours, in order to minimize any unwanted side effects.

An effective dose of a inhibitor of the present invention may also be administered using a weekly, biweekly or triweekly dosing period. The dosing period may be restricted to, e.g., 8 weeks, 12 weeks or until clinical progression has been established. As non-limiting examples, treatment according to the present invention may be provided as a daily dosage of a inhibitor of the present invention in an amount of about 0.1-100 mg/kg, such as 0.2, 0.5, 0.9, 1.0, 1.1, 1.5, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 45, 50, 60, 70, 80, 90 or 100 mg/kg, per day, on at least one of days 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40, or alternatively, at least one of weeks 1, 2, 3, 4, 5, 6, 7, 8, 9, 10, 11, 12, 13, 14, 15, 16, 17, 18, 19 or 20 after initiation of treatment, or any combination thereof, using single or divided doses every 24, 12, 8, 6, 4, or 2 hours, or any combination thereof.

As used herein, the term “pharmaceutical composition” refers to a composition described herein, or pharmaceutically acceptable salts thereof, with other agents such as carriers and/or excipients. The pharmaceutical compositions as provided herewith typically include a pharmaceutically acceptable carrier.

As used herein, the term “pharmaceutically acceptable carrier” includes any and all solvents, diluents, or other liquid vehicle, dispersion or suspension aids, surface active agents, isotonic agents, thickening or emulsifying agents, preservatives, solid binders, lubricants and the like, as suited to the particular dosage form desired. Remington's Pharmaceutical-Sciences, Sixteenth Edition, E. W. Martin (Mack Publishing Co., Easton, Pa., 1980) discloses various carriers used in formulating pharmaceutical compositions and known techniques for the preparation thereof.

### Host Immune Cells of the Present Invention:

The first object of the present invention relates to a host immune cell engineering to express a) a chimeric antigen receptor (CAR) that binds to CD38, wherein binding of the CAR to CD38 is capable of delivering an activation signal to the host immune cell, and b) a chimeric co-stimulating receptor (CCR) that binds to a second antigen, wherein binding of the CCR to the second antigen is capable of delivering a costimulatory signal to the host immune cell but does not alone deliver an activation signal to the host immune cell, wherein the host immune cell is capable of (i) exhibiting negligible cytotoxicity against cells that are single positive for CD38, and (ii) inducing cytotoxicity against cells that are positive for both CD38 and the second antigen.

### Host Immune Cells:

In some embodiments, the host immune cell is a hematopoietic cell from the lymphoid lineage that comprises B, T and natural killer (NK) cells. In some embodiments, the host immune cell is selected from the group consisting of a T cell, a Natural Killer (NK) cell, a cytotoxic T lymphocyte (CTL), a human embryonic stem cell, and a pluripotent stem cell from which lymphoid cells may be differentiated.

In some embodiments, the host cell is a pluripotent stem cell (PSC). PSCs can be indeed be modified by a CAR and then can be used for deriving T cells (e.g. WO 2017100403). PSCs include embryonic stem cell (ESCs) and induced pluripotent stem cell (iPSCs). iPSCs can be generated directly from adult cells (e.g., somatic cells). iPSCs can be typically derived or generated by introducing a specific set of pluripotency-associated genes, or “reprogramming factors”, into a given cell type. Reprogramming factors include, but are not limited to, OCT4 (also known as “POU5FL”), SOX2, cMYC, and KLF4, which are also known as Yamanaka factors. See Takahashi, K; Yamanaka, S (2006). “Induction of pluripotent stem cells from mouse embryonic and adult fibroblast cultures by defined factors”. Cell 126 (4): 663-76.

In some embodiments, the host cell is a hematopoietic stem cell. As used herein, the term “hematopoietic stem cell” or “HSC” refers to blood cells that have the capacity to self-renew and to differentiate into precursors of blood cells. These precursor cells are immature blood cells that cannot self-renew and must differentiate into mature blood cells. Hematopoietic stem progenitor cells display a number of phenotypes, such as Lin-CD34+CD38−CD90+CD45RA−, Lin-CD34+CD38−CD90−CD45RA−, Lin-CD34+CD38+IL-3aloCD45RA−, and Lin-CD34+CD38+CD10+(Daley et al., Focus 18:62-67, 1996; Pimentel, E., Ed., Handbook of Growth Factors Vol. III: Hematopoietic Growth Factors and Cytokines, pp. 1-2, CRC Press, Boca Raton, Fla., 1994). Within the bone marrow microenvironment, the stem cells self-renew and maintain continuous production of hematopoietic stem cells that give rise to all mature blood cells throughout life. In some embodiments, the hematopoietic progenitor cells or hematopoietic stem cells are isolated form peripheral blood cells.

Chimeric anti gen receptor (CAR) The chimeric antigen receptor (CAR) typically comprises an extracellular domain and an intracellular domain joined by a transmembrane domain.

The extracellular domain, expressed on the surface of the host immune cell, comprises an antigen binding domain having binding affinity for CD38.

In some embodiments, such antigen binding domain is an antibody, preferably a single chain antibody. Preferably, the antibody is a humanized antibody. Particularly, such antigen binding domain is an antibody fragment selected from fragment antigen binding (Fab) fragments, F(ab′)2 fragments, Fab′ fragments, Fv fragments, recombinant IgG (rlgG) fragments, single chain antibody fragments, single chain variable fragments (scFv), single domain antibodies (e.g., sdAb, sdFv, nanobody) fragments, diabodies, and multi-specific antibodies formed from antibody fragments. In some embodiments, the antibodies are single-chain antibody fragments comprising a variable heavy chain region and/or a variable light chain region, such as scFv. Particularly, such antigen binding domain is selected from a Fab and a scFv.

In some embodiments, when the antigen targeting domain is a scFv, the scFv can be derived from the variable heavy chain (VH) and variable light chain (VL) regions of an antigen-specific mAb linked by a flexible linker. The scFv retains the same specificity and a similar affinity as the full antibody from which it is derived. The peptide linker connecting scFv VH and VL domains joins the carboxyl terminus of one variable region domain to the amino terminus of the other variable domain without compromising the fidelity of the VH-VL paring and antigen-binding sites. Peptide linkers can vary from 10 to 30 amino acids in length. In some embodiments, the scFv peptide linker is a Gly/Ser linker.

In some embodiments, the scFv is specific for an epitope located in the extracellular domain of CD38.

In some embodiments, the scFv comprises a VH domain comprising i) the H-CDR1 as set forth in SEQ ID NO:3, ii) the H-CDR2 as set forth in SEQ ID NO:4 and iii) the H-CDR3 as set forth in SEQ ID NO:5, and, a VL domain comprising i) the L-CDR1 as set forth in SEQ ID NO:6, ii) the L-CDR2 as set forth in SEQ ID NO:7 and iii) the L-CDR3 as set forth in SEQ ID NO:8.

SEQ ID NO: 3 (H-CDR1): GYTFTSYW
SEQ ID NO: 4 (H-CDR2): IYPGDGDT
SEQ ID NO: 5 (H-CDR3): ARERTTGAPRYFDV
SEQ ID NO: 6 (L-CDR1): ENIYSF
SEQ ID NO: 7 (L-CDR2): NTK
SEQ ID NO: 8 (L-CDR3): QHHYGIPLT

In some embodiments, the scFv comprises a VH domain having at least 90% of identity with the amino acid sequence as set forth in SEQ ID NO:9.

In some embodiments, the scFv comprises a VL domain having at least 90% of identity with the amino acid sequence as set forth in SEQ ID NO:10.

> IgH VH1.87-D1.1-J1:
SEQ ID NO: 9
QVQLQQSGAELARPGASVKLSCKASGYTFTSYWMQWVKQRPG
QGLEWIGAIYPGDGDTRYTQKFKGKATLTADKSSSTAYMQLS
NLTSEDSAVYYCARERTTGAPRYFDVWGAGTTVTVSS
>Igk Vk12.44-Jk5:
SEQ ID NO: 10
DIQMTQSPASLSASVGETVTITCRASENIYSFLAWYQQKQGK
SPQLLVYNTKTLTEGVPSRFSGSGSGTQFSLKINNLQPEDFG
SYYCQHHYGIPLTFGAGTKLELK

In some embodiments, the scFv comprises an amino acid sequence having 90% of identity with the amino acid sequence as set forth in SEQ NO:11.

> anti-CD38 scFv
SEQ ID NO: 11
DIQMTQSPASLSASVGETVTITCRASENIYSFLAWYQQKQGKSPQL
LVYNTKTLTEGVPSRFSGSGSGTQFSLKINNLQPEDFGSYYCQHHY
GIPLTFGAGTKLELKGGGGSGGGGSGGGGSQVQLQQSGAELARPGA
SVKLSCKASGYTFTSYWMQWVKQRPGQGLEWIGAIYPGDGDTRYTQ
KFKGKATLTADKSSSTAYMQLSNLTSEDSAVYYCARERTTGAPRYF
DVWGAGTTVTVSS

The transmembrane domain is typically a hydrophobic alpha helix that spans across the lipid bilayer of the cell membrane. The transmembrane domain of the CAR thus functions to anchor the extracellular domain on the cell surface. In some embodiments, the transmembrane domain is derived either from a natural or from a synthetic source. Where the source is natural, the domain in some embodiments is derived from any membrane-bound or transmembrane protein. Transmembrane regions include those derived from (i.e. comprise at least the transmembrane region(s) of) the alpha, beta or zeta chain of the T-cell receptor, CD28, CD3 zeta, CD3 epsilon, CD3 gamma, CD3 delta, CD45, CD4, CD5, CD8, CD9, CD16, CD22, CD33, CD37, CD64, CD80, CD86, CD134, CD137, CD154, ICOS/CD278, GITR/CD357, NKG2D, and DAP molecules. Alternatively, the transmembrane domain in some embodiments is synthetic. In some embodiments, the synthetic transmembrane domain comprises predominantly hydrophobic residues such as leucine and valine. In some embodiments, a triplet of phenylalanine, tryptophan and valine will be found at each end of a synthetic transmembrane domain. A transmembrane domain is thermodynamically stable in a membrane. It may be a single alpha helix, a transmembrane beta barrel, a beta-helix of gramicidin A, or any other structure. Optionally, a short oligo- or polypeptide linker, preferably between 2 and 10 amino acids in length may form the linkage between the transmembrane domain and the intracellular signalling domain(s) of the CAR. A glycine-serine doublet may provide a suitable linker.

In some embodiments, the transmembrane domain comprises the amino acid sequence having at 90% of identity with the amino acid sequence as set forth in SEQ ID NO:12.

> CD8 transmembrane domain
SEQ ID NO: 12
HFVPVFLPAKPTTTPAPRPPTPAPTIASQPLSLRPEACRPAAGGAV
HTRGLDFACDIYIWAPLAGTCGVLLLSLVITLYCNHRN

The intracellular domain of the CAR is composed of an intracellular signalling domain. The role of the intracellular signalling domain of the CAR is to produce an activation signal to the host immune cell as soon as the extracellular domain has recognized the antigen (i.e. CD38). In particular, the intracellular signalling domain of the CAR triggers or elicits activation of at least one of the normal effector functions of the host immune cell. Examples of intracellular signalling domain sequences that are of particular use in the invention include those derived from an intracellular signalling domain of a lymphocyte receptor chain, a TCR/CD3 complex protein, an Fc receptor subunit, an IL-2 receptor subunit, CD3ζ, FcRγ, FcRβ, CD3γ, CD3δ, CD3ε, CD5, CD22, CD79a, CD79b, CD66d, CD278(ICOS), FcsRI, DAP10, and DAP12. It is particularly preferred that the intracellular signalling domain in the CAR comprises a cytoplasmic signalling sequence derived from CD3ζ.

In some embodiments, the intracellular domain in the CAR comprises an amino acid sequence having at least 90% of identity with the amino acid sequence as set forth in SEQ ID NO:13.

> CD3ζ signalling domain
SEQ ID NO: 13
RVKFSRSADAPAYQQGQNQLYNELNLGRREEYDVLDKRRGRDPEMG
GKPQRRKNPQEGLYNELQKDKMAEAYSEIGMKGERRRGKGHDGLYQ
GLSTATKDTYDALHMQALPPR

In some embodiments, the CAR of the present invention comprises an amino acid sequence having at least 90% of identity with the amino acid sequence as set forth in SEQ ID NO:14.

> anti-CD38 CAR
SEQ ID NO: 14
DIQMTQSPASLSASVGETVTITCRASENIYSFLAWYQQKQGKSPQLLVYNTKTLTEGVPSRFSGSGSGT
QFSLKINNLQPEDFGSYYCQHHYGIPLTFGAGTKLELKGGGGSGGGGSGGGGSQVQLQQSGAELARPGA
SVKLSCKASGYTFTSYWMQWVKQRPGQGLEWIGAIYPGDGDTRYTQKFKGKATLTADKSSSTAYMQLSN
LTSEDSAVYYCARERTTGAPRYFDVWGAGTTVTVSSLEHFVPVFLPAKPTTTPAPRPPTPAPTIASQPL
SLRPEACRPAAGGAVHTRGLDFACDIYIWAPLAGTCGVLLLSLVITLYCNHRNRVKFSRSADAPAYQQG
QNQLYNELNLGRREEYDVLDKRRGRDPEMGGKPQRRKNPQEGLYNELQKDKMAEAYSEIGMKGERRRGK
GHDGLYQGLSTATKDTYDALHMQALPPR

Chimeric co-stimulating receptor (CCR):

The chimeric co-stimulating receptor (CCR) typically comprises an extracellular domain and an intracellular domain joined by a transmembrane domain.

The extracellular domain, expressed on the surface of the host immune cell, comprises an antigen binding domain having a binding affinity for second antigen.

In some embodiments, the antigen binding domain has a binding affinity for a second antigen selected from the group consisting of G-Protein Coupled Receptor 5D (GPRC5D), CD138, NY-ESO1, CD19 and SLAMF7/CS1. More particularly, the antigen binding domain has a binding affinity for SLAMF7/CS1.

In some embodiments, such antigen binding domain is an antibody, preferably a single chain antibody. Preferably, the antibody is a humanized antibody. Particularly, such antigen binding domain is an antibody fragment selected from fragment antigen binding (Fab) fragments, F(ab′)2 fragments, Fab′ fragments, Fv fragments, recombinant IgG (rlgG) fragments, single chain antibody fragments, single chain variable fragments (scFv), single domain antibodies (e.g., sdAb, sdFv, nanobody) fragments, diabodies, and multi-specific antibodies formed from antibody fragments. In some embodiments, the antibodies are single-chain antibody fragments comprising a variable heavy chain region and/or a variable light chain region, such as scFv.

Particularly, such antigen binding domain is selected from a Fab and a scFv. In some embodiments, when the antigen targeting domain is a scFv In some embodiments, the scFv is specific for an epitope located in the extracellular domain of SLAMF7.

In some embodiments, the scFv comprises a VH domain having at least 90% of identity with the amino acid sequence as set forth in SEQ ID NO:15.

In some embodiments, the scFv comprises a VL domain having at least 90% of identity with the amino acid sequence as set forth in SEQ ID NO:16.

> VH domain of the anti-SLAMF7
antibody
SEQ ID NO: 15
DIVMTQSQKSMSTSVGDRVSITCKASQDVITGVAWYQQKPGQSPK
LLIYSASYRYTGVPDRFTGSGSGTDFTFTISNVQAEDLAVYYCQQ
HYSTPLTFGAGTKLELK
>VL domain of the anti-SLAMF7
antibody
SEQ ID NO: 16
SQVQLQQPGAELVRPGASVKLSCKASGYSFTTYWMNWVKQRPGQGL
EWIGMIHPSDSETRLNQKFKDKATLTVDKSSSTAYMQLSSPTSEDS
AVYYCARSTMIATRAMDYWGQGTSVTVS

In some embodiments, the scFv comprises an amino acid sequence having 90% of identity with the amino acid sequence as set forth in SEQ ID NO:17.

> anti-SLAMF7 scFV
SEQ ID NO: 17
SQVQLQQPGAELVRPGASVKLSCKASGYSFTTYWMNWVKQRPGQGL
EWIGMIHPSDSETRLNQKFKDKATLTVDKSSSTAYMQLSSPTSEDSA
VYYCARSTMIATRAMDYWGQGTSVTVSGGGGSGGGGSGGGGSDIVMT
QSQKSMSTSVGDRVSITCKASQDVITGVAWYQQKPGQSPKLLIYSAS
YRYTGVPDRFTGSGSGTDFTFTISNVQAEDLAVYYCQQHYSTPLTFG
AGTKLELK

The transmembrane domain of the CCR is the same nature as for the CAR and is typically a hydrophobic alpha helix that spans across the lipid bilayer of the cell membrane. In some embodiments, the transmembrane domain is derived either from a natural or from a synthetic source. Where the source is natural, the domain in some embodiments is derived from any membrane-bound or transmembrane protein. Transmembrane regions include those derived from (i.e. comprise at least the transmembrane region(s) of) the alpha, beta or zeta chain of the T-cell receptor, CD28, CD3 zeta, CD3 epsilon, CD3 gamma, CD3 delta, CD45, CD4, CD5, CD8, CD9, CD16, CD22, CD33, CD37, CD64, CD80, CD86, CD134, CD137, CD154, ICOS/CD278, GITR/CD357, NKG2D, and DAP molecules. Alternatively, the transmembrane domain in some embodiments is synthetic. In some embodiments, the synthetic transmembrane domain comprises predominantly hydrophobic residues such as leucine and valine. In some embodiments, a triplet of phenylalanine, tryptophan and valine will be found at each end of a synthetic transmembrane domain. A transmembrane domain is thermodynamically stable in a membrane. It may be a single alpha helix, a transmembrane beta barrel, a beta-helix of gramicidin A, or any other structure. Optionally, a short oligo- or polypeptide linker, preferably between 2 and 10 amino acids in length may form the linkage between the transmembrane domain and the intracellular signalling domain(s) of the CCR. A glycine-serine doublet may provide a suitable linker.

In some embodiments, the transmembrane domain comprises the amino acid sequence having at 90% of identity with the amino acid sequence as set forth in SEQ ID NO:12.

The intracellular domain of the CCR is composed of an intracellular signalling domain. Contrary to the CAR, the role of the intracellular signalling domain of the CCR is not able to produce an activation signal per se to the host immune cell as soon as the extracellular domain has recognized the antigen (e.g. SLAMF7). The role of the intracellular signalling domain of the CCR is thus to deliver a costimulatory signal to the host immune cell but does not alone deliver an activation signal to the host immune cell. Accordingly, the intracellular signalling domain of the CCR comprises one or more intracellular domain(s) of a costimulatory molecule.

A costimulatory molecule can be defined as a cell surface molecule that is required for an efficient response of lymphocytes to an antigen. Examples of such molecules include CD27, CD28, 4-1BB (CD137), OX40 (CD134), CD30, CD40, CD244 (2B4), ICOS, lymphocyte function-associated antigen-1 (LFA-1), CD2, CD7, LIGHT, NKG2C, B7-H3, and a ligand that specifically binds with CD83, CD8, CD4, b2c, CD80, CD86, DAP10, DAP12, MyD88, BTNL3, and NKG2D. The intracellular signalling portion of the above recited co-stimulatory domains can be used alone or in combination with other co-stimulatory domains. In particular, the CCR can comprise one or more co-stimulatory domains from the group consisting of CD27, CD28, 4-1BB (CD137), OX40 (CD134), CD30, CD40, CD244 (2B4), ICOS, LFA-1, CD2, CD7, LIGHT, NKG2C, B7-H3, and a ligand that specifically binds with CD83, CD8, CD4, b2c, CD80, CD86, DAP10, DAP12, MyD88, BTNL3, and NKG2D.

In some embodiments, the intracellular domain of the CCR comprises the co-stimulatory domain of 4-1BB (CD137).

In some embodiments, the intracellular domain of the CCR comprises an amino acid sequence having at least 90% of identity with the amino acid sequence as set forth in SEQ ID NO:18.

> CD137 costimulating domain
SEQ ID NO: 18
KRGRKKLLYIFKQPFMRPVQTTQEEDGCSCRFPEEEEGGCEL

In some embodiments, the CCR of the present invention comprises an amino acid sequence having at least 90% of identity with the amino acid sequence as set forth in SEQ ID NO:19.

> anti-SLAMF7 CCR
SEQ ID NO: 19
SQVQLQQPGAELVRPGASVKLSCKASGYSFTTYWMNWVKQRPGQGLEWIGMIHPSDSETRLNQKFKDKA
TLTVDKSSSTAYMQLSSPTSEDSAVYYCARSTMIATRAMDYWGQGTSVTVSGGGGSGGGGSGGGGSDIV
MTQSQKSMSTSVGDRVSITCKASQDVITGVAWYQQKPGQSPKLLIYSASYRYTGVPDRFTGSGSGTDFT
FTISNVQAEDLAVYYCQQHYSTPLTFGAGTKLELKLEHFVPVFLPAKPTTTPAPRPPTPAPTIASQPLS
LRPEACRPAAGGAVHTRGLDFACDIYIWAPLAGTCGVLLLSLVITLYCNHRNMH

### Methods for Preparing the Host Immune Cells of the Present Invention:

The host immune cell of the present is preparing by any conventional method well known in the art. Typically, the host immune cells are transduced in order to express a polynucleotide that encodes for the CAR and a polynucleotide that encodes for the CCR.

Thus, a further object of the present invention relates to a method of preparing a host immune cell of the present invention, comprising the steps consisting of introducing into a host immune cell i) a polynucleotide that encodes for the CAR and ii) a polynucleotide that encodes for the CCR.

Use of bicistronic polynucleotides or vectors encoding for both the CAR and the CCR are particularly suitable for preparing the host immune cells of the present invention. As used herein the term “bicistronic” refers to a polynucleotide or vector that comprises two cistrons, i.e. comprising two genes. There are several strategies which can be employed to construct bicistronic or multicistronic vectors including, but not limited to, (1) multiple promoters fused to the open reading frames for the CAR and CCR; (2) insertion of splicing signals between units of CAR and CCR; fusion of the CAR and CCR whose expressions are driven by a single promoter; (3) insertion of proteolytic cleavage sites between the CAR and CCR (self-cleavage peptide); and (iv) insertion of internal ribosomal entry sites (IRESs). In some embodiments, the CAR and CCR are expressed in a single open reading frame (ORF), thereby creating a single polypeptide. In this embodiment, an amino acid sequence or linker containing a high efficiency cleavage site is disposed between the CAR unit and the CCR unit. As used herein, “high cleavage efficiency” is defined as more than 50%, more than 70%, more than 80%, or more than 90% of the translated protein is cleaved. Cleavage efficiency may be measured by Western Blot analysis. Examples of high efficiency cleavage sites include porcine teschovirus-1 2A (P2A), FMDV 2A (abbreviated herein as F2A); equine rhinitis A virus (ERAV) 2A (E2A); and Thoseaasigna virus 2A (T2A), cytoplasmic polyhedrosis virus 2A (BmCPV2A) and flacherie Virus 2A (BmIFV2A), or a combination thereof. In some embodiments, the high efficiency cleavage site is P2A. High efficiency cleavage sites are described in Kim J H, Lee S R, Li L H, Park H J, Park J H, Lee K Y, et al. (2011) High Cleavage Efficiency of a 2A Peptide Derived from Porcine Teschovirus-1 in Human Cell Lines, Zebrafish and Mice. PLoS ONE 6(4): e18556, the contents of which are incorporated herein by reference.

In some embodiments, the host immune cell is transduced with a polynucleotide that encodes for the amino acid sequence as set forth in SEQ ID NO:20.

SEQ ID NO: 20
SQVQLQQPGAELVRPGASVKLSCKASGYSFTTYWMNWVKQRPGQGLEWIGMIHPSDSETRLNQKFKDKA
TLTVDKSSSTAYMQLSSPTSEDSAVYYCARSTMIATRAMDYWGQGTSVTVSGGGGSGGGGSGGGGSDIV
MTQSQKSMSTSVGDRVSITCKASQDVITGVAWYQQKPGQSPKLLIYSASYRYTGVPDRFTGSGSGTDFT
FTISNVQAEDLAVYYCQQHYSTPLTFGAGTKLELKLEHFVPVFLPAKPTTTPAPRPPTPAPTIASQPLS
LRPEACRPAAGGAVHTRGLDFACDIYIWAPLAGTCGVLLLSLVITLYCNHRNMHKRGRKKLLYIFKQPF
MRPVQTTQEEDGCSCRFPEEEEGGCELGSGGSGATNFSLLKQAGDVEENPGPQRPGERVMALPVTALLL
PLALLLHAARPDIQMTQSPASLSASVGETVTITCRASENIYSFLAWYQQKQGKSPQLLVYNTKTLTEGV
PSRFSGSGSGTQFSLKINNLQPEDFGSYYCQHHYGIPLTFGAGTKLELKGGGGSGGGGSGGGGSQVQLQ
QSGAELARPGASVKLSCKASGYTFTSYWMQWVKQRPGQGLEWIGAIYPGDGDTRYTQKFKGKATLTADK
SSSTAYMQLSNLTSEDSAVYYCARERTTGAPRYFDVWGAGTTVTVSSLEHFVPVFLPAKPTTTPAPRPP
TPAPTIASQPLSLRPEACRPAAGGAVHTRGLDFACDIYIWAPLAGTCGVLLLSLVITLYCNHRNRVKES
RSADAPAYQQGQNQLYNELNLGRREEYDVLDKRRGRDPEMGGKPQRRKNPQEGLYNELQKDKMAEAYSE
IGMKGERRRGKGHDGLYQGLSTATKDTYDALHMQALPPR

In some embodiments, the polynucleotide comprises a nucleic acid sequence having at least 90% of identity with the nucleic acid sequence as set forth in SEQ ID NO:21.

SEQ ID NO: 21
>TCCCAGGTCCAACTGCAGCAGCCTGGGGCTGAGCTGGTGAGGCCTGGAGCTTCAGTGAAGCTGTCCTGC
AAGGCTTCGGGGTACTCCTTCACCACCTACTGGATGAACTGGGTGAAGCAGAGGCCTGGACAAGGCCTT
GAGTGGATTGGCATGATTCATCCTTCCGATAGTGAAACTAGGTTAAATCAGAAGTTCAAGGACAAGGCC
ACATTGACTGTAGACAAATCCTCCAGCACAGCCTACATGCAACTCAGCAGCCCGACATCTGAGGACTCT
GCGGTCTATTACTGTGCAAGATCTACTATGATTGCGACGAGGGCTATGGACTACTGGGGTCAAGGAACC
TCAGTCACCGTCTCCGGCGGTGGCGGTTCTGGTGGCGGTGGCTCCGGCGGTGGCGGTTCTGACATTGTG
ATGACCCAGTCTCAGAAATCCATGTCCACATCAGTAGGAGACAGGGTCAGCATCACCTGCAAGGCCAGT
CAGGATGTTATTACTGGTGTAGCCTGGTATCAACAGAAACCAGGGCAATCTCCTAAATTACTGATTTAC
TCGGCATCCTACCGGTACACTGGAGTCCCTGATCGCTTCACTGGCAGTGGATCTGGGACGGATTTCACT
TTCACCATCAGCAATGTGCAGGCTGAAGACCTGGCAGTTTATTACTGTCAGCAACATTATAGTACTCCT
CTCACTTTCGGTGCTGGGACCAAGCTGGAGCTGAAACTCGAGCACTTCGTGCCGGTCTTCCTGCCAGCG
AAGCCCACCACGACGCCAGCGCCGCGACCACCAACACCGGCGCCCACCATCGCGTCGCAGCCCCTGTCC
CTGCGCCCAGAGGCGTGCCGGCCAGCGGCGGGGGGCGCAGTGCACACGAGGGGGCTGGACTTCGCCTGT
GATATCTACATCTGGGCGCCCTTGGCCGGGACTTGTGGGGTCCTTCTCCTGTCACTGGTTATCACCCTT
TACTGCAACCACAGGAACATGCATAAACGGGGCAGAAAGAAACTCCTGTATATATTCAAACAACCATTT
ATGAGACCAGTACAAACTACTCAAGAGGAAGATGGCTGTAGCTGCCGATTTCCAGAAGAAGAAGAAGGA
GGATGTGAACTGGGCTCCGGAGGATCTGGAGCAACAAACTTCTCACTACTCAAACAAGCAGGTGACGTG
GAGGAGAATCCCGGCCCCCAGCGTCCTGGGGAGCGCGTCATGGCCTTACCAGTGACCGCCTTGCTCCTG
CCGCTGGCCTTGCTGCTCCACGCCGCCAGGCCGGACATCCAGATGACTCAGTCTCCAGCCTCCCTATCT
GCATCTGTGGGGGAAACTGTCACCATCACATGTCGAGCAAGTGAGAATATTTACAGTTTTTTAGCATGG
TATCAGCAGAAACAGGGAAAATCTCCTCAGCTCCTGGTCTATAATACAAAAACCTTAACAGAAGGTGTG
CCATCAAGGTTCAGTGGCAGTGGATCAGGCACACAGTTTTCTCTGAAGATTAATAACCTGCAGCCTGAG
GATTTTGGGAGTTATTACTGTCAACATCATTATGGTATTCCACTCACGTTCGGTGCTGGGACCAAGCTG
GAGCTGAAAGGCGGTGGCGGTTCTGGTGGCGGTGGCTCCGGCGGTGGCGGTTCTCAGGTTCAGCTCCAG
CAGTCTGGGGCTGAGCTGGCAAGACCTGGGGCTTCAGTGAAGTTGTCCTGCAAGGCTTCTGGCTACACC
TTTACTTCCTACTGGATGCAGTGGGTAAAACAGAGGCCTGGACAGGGTCTGGAATGGATTGGGGCTATT
TATCCTGGAGATGGTGATACTAGGTACACTCAGAAGTTCAAGGGCAAGGCCACATTGACTGCAGATAAA
TCCTCCAGCACAGCCTACATGCAACTCAGCAACTTGACATCTGAGGACTCTGCGGTCTATTACTGTGCA
AGAGAGAGGACTACGGGAGCCCCACGGTACTTCGATGTCTGGGGCGCAGGGACCACGGTCACCGTCTCC
TCACTCGAGCACTTCGTGCCGGTCTTCCTGCCAGCGAAGCCCACCACGACGCCAGCGCCGCGACCACCA
ACACCGGCGCCCACCATCGCGTCGCAGCCCCTGTCCCTGCGCCCAGAGGCGTGCCGGCCAGCGGCGGGG
GGCGCAGTGCACACGAGGGGGCTGGACTTCGCCTGTGATATCTACATCTGGGCGCCCTTGGCCGGGACT
TGTGGGGTCCTTCTCCTGTCACTGGTTATCACCCTTTACTGCAACCACAGGAACAGAGTGAAGTTCAGC
AGGAGCGCAGACGCCCCCGCGTACCAGCAGGGCCAGAACCAGCTCTATAACGAGCTCAATCTAGGACGA
AGAGAGGAGTACGATGTTTTGGACAAGAGACGTGGCCGGGACCCTGAGATGGGGGGAAAGCCGCAGAGA
AGGAAGAACCCTCAGGAAGGCCTGTACAATGAACTGCAGAAAGATAAGATGGCGGAGGCCTACAGTGAG
ATTGGGATGAAAGGCGAGCGCCGGAGGGGCAAGGGGCACGATGGCCTTTACCAGGGTCTCAGTACAGCC
ACCAAGGACACCTACGACGCCCTTCACATGCAGGCCCTGCCCCCTCGCTAA

It is contemplated that the polynucleotide construct can be introduced into the host immune cells as naked DNA or in a suitable vector. Physical methods for introducing a polynucleotide construct into a host cell include calcium phosphate precipitation, lipofection, particle bombardment, microinjection, electroporation, and the like. Other means can be used including colloidal dispersion systems, such as macromolecule complexes, nanocapsules, microspheres, beads, and lipid-based systems including oil-in-water emulsions, micelles, mixed micelles, and liposomes. In some embodiments, the polynucleotide construct is introduced into the host immune cell by a viral vector that is an adeno-associated virus (AAV), a retrovirus, lentivirus, bovine papilloma virus, an adenovirus vector, a vaccinia virus, a polyoma virus, or an infective virus. In some embodiments, the vector is a retroviral. Retroviruses may be chosen as gene delivery vectors due to their ability to integrate their genes into the host genome, transferring a large amount of foreign genetic material, infecting a broad spectrum of species and cell types and for being packaged in special cell-lines. In order to construct a retroviral vector, the polynucleotide is inserted into the viral genome in the place of certain viral sequences to produce a virus that is replication-defective. In order to produce virions, a packaging cell line is constructed containing the gag, pol, and/or env genes but without the LTR and/or packaging components. When a recombinant plasmid containing a cDNA, together with the retroviral LTR and packaging sequences is introduced into this cell line (by calcium phosphate precipitation for example), the packaging sequence allows the RNA transcript of the recombinant plasmid to be packaged into viral particles, which are then secreted into the culture media. The media containing the recombinant retroviruses is then collected, optionally concentrated, and used for gene transfer. Retroviral vectors are able to infect a broad variety of cell types. Lentiviruses are complex retroviruses, which, in addition to the common retroviral genes gag, pol, and env, contain other genes with regulatory or structural function. The higher complexity enables the virus to modulate its life cycle, as in the course of latent infection. Some examples of lentivirus include the Human Immunodeficiency Viruses (HIV 1, HIV 2) and the Simian Immunodeficiency Virus (SIV). Lentiviral vectors have been generated by multiply attenuating the HIV virulence genes, for example, the genes env, vif, vpr, vpu and nef are deleted making the vector biologically safe. Lentiviral vectors are known in the art, see, e.g. U.S. Pat. Nos. 6,013,516 and 5,994,136, both of which are incorporated herein by reference. In general, the vectors are plasmid-based or virus-based, and are configured to carry the essential sequences for incorporating foreign nucleic acid, for selection and for transfer of the nucleic acid into a host cell. The gag, pol and env genes of the vectors of interest also are known in the art. Thus, the relevant genes are cloned into the selected vector and then used to transform the target cell of interest. Recombinant lentivirus capable of infecting a non-dividing cell wherein a suitable host cell is transfected with two or more vectors carrying the packaging functions, namely gag, pol and env, as well as rev and tat is described in U.S. Pat. No. 5,994,136, incorporated herein by reference. This describes a first vector that can provide a nucleic acid encoding a viral gag and a pol gene and another vector that can provide a nucleic acid encoding a viral env to produce a packaging cell. Introducing a vector providing a heterologous gene into that packaging cell yields a producer cell which releases infectious viral particles carrying the foreign gene of interest. The env preferably is an amphotropic envelope protein which allows transduction of cells of human and other species. Typically, the vector of the present invention include “control sequences'”, which refers collectively to promoter sequences, polyadenylation signals, transcription termination sequences, upstream regulatory domains, origins of replication, internal ribosome entry sites (“IRES”), enhancers, and the like, which collectively provide for the replication, transcription and translation of a coding sequence in a recipient cell. Not all of these control sequences need always be present so long as the selected coding sequence is capable of being replicated, transcribed and translated in an appropriate host cell. Another nucleic acid sequence, is a “promoter” sequence, which is used herein in its ordinary sense to refer to a nucleotide region comprising a DNA regulatory sequence, wherein the regulatory sequence is derived from a gene which is capable of binding RNA polymerase and initiating transcription of a downstream (3′-direction) coding sequence. Transcription promoters can include “inducible promoters” (where expression of a polynucleotide sequence operably linked to the promoter is induced by an analyte, cofactor, regulatory protein, etc.), “repressible promoters” (where expression of a polynucleotide sequence operably linked to the promoter is induced by an analyte, cofactor, regulatory protein, etc.), and “constitutive promoters”. In some embodiments, the polynucleotide is encoded by a nucleic acid molecule whose sequence has been codon optimized for expression in a mammalian cell. Codon optimization refers to the discovery that the frequency of occurrence of synonymous codons (i.e., codons that code for the same amino acid) in coding DNA is biased in different species. Such codon degeneracy allows an identical polypeptide to be encoded by a variety of nucleotide sequences. A variety of codon optimization methods is known in the art, and include, e.g., methods disclosed in at least U.S. Pat. Nos. 5,786,464 and 6,114,148. In order to confirm the presence of the polynucleotide that encodes for both the CAR and CCR in the host immune cell, a variety of assays may be performed. Such assays include, for example, “molecular biological” assays well known such as Southern and Northern blotting, RT-PCR and quantitative PCR; or “biochemical” assays, such as detecting the presence or absence of a particular peptide.

### CD38 Gene Edition:

In some embodiments, the host immune cell of the present invention is engineered such that it does not express CD38.

In some embodiments, the method includes introducing into the host immune cell a genome-editing nuclease designed to edit the CD38 coding region, and culturing the host immune cell under conditions for the genome-editing nuclease to modify the CD38 coding region to inhibit the expression of CD38. In some embodiments, introducing the genome-editing nuclease into the host immune cell includes introducing into the host immune cell a polynucleotide that encodes the genome-editing nuclease. In some embodiments, the genome-editing nuclease includes a TALEN nuclease, a CRISPR-associated endonuclease, or a megaTAL nuclease.

In some embodiments, the genome-editing nuclease is a CRISPR-associated endonuclease. CRISPR/Cas systems for gene editing in eukaryotic cells typically involve (1) a guide RNA molecule (gRNA) comprising a targeting sequence (which is capable of hybridizing to the genomic DNA target sequence), and sequence which is capable of encoding for the CRISPR-associated endonuclease.

In some embodiments, the CRISPR-associated endonuclease is a Cas9 nuclease. The Cas9 nuclease can have a nucleotide sequence identical to the wild type Streptococcus pyrogenes sequence. In some embodiments, the CRISPR-associated endonuclease can be a sequence from other species, for example other Streptococcus species, such as thermophilus; Pseudomona aeruginosa, Escherichia coli, or other sequenced bacteria genomes and archaea, or other prokaryotic microorganisms. Alternatively, the wild type Streptococcus pyogenes Cas9 sequence can be modified. The nucleic acid sequence can be codon optimized for efficient expression in mammalian cells, i.e., “humanized.” A humanized Cas9 nuclease sequence can be for example, the Cas9 nuclease sequence encoded by any of the expression vectors listed in Genbank accession numbers KM099231.1 GL669193757; KM099232.1 GL669193761; or KM099233.1 GL669193765. Alternatively, the Cas9 nuclease sequence can be for example, the sequence contained within a commercially available vector such as pX330, pX260 or pMJ920 from Addgene (Cambridge, MA). In some embodiments, the Cas9 endonuclease can have an amino acid sequence that is a variant or a fragment of any of the Cas9 endonuclease sequences of Genbank accession numbers KM099231.1 GL669193757; KM099232.1; GL669193761; or KM099233.1 GL669193765 or Cas9 amino acid sequence of pX330, pX260 or pMJ920 (Addgene, Cambridge, MA). Artificial CRISPR/Cas systems can be generated, using technology known in the art, e.g., that are described in U.S. Publication No. 20140068797, WO2015/048577, and Cong (2013) Science 339: 819-823. Other artificial CRISPR/Cas systems that are known in the art may also be generated, e.g., that described in Tsai (2014) Nature Biotechnol., 32:6 569-576, U.S. Pat. Nos. 8,871,445; 8,865,406; 8,795,965; 8,771,945; and 8,697,359, the contents of which are hereby incorporated by reference in their entirety. Such systems can be generated by, for example, engineering a CRISPR/Cas system to include a gRNA molecule comprising a targeting sequence that hybridizes to a sequence of the CD38 gene. In some embodiments, the gRNA comprises a targeting sequence which is fully complementarity to 15-25 nucleotides, e.g., 20 nucleotides, of the CD38 gene. In some embodiments, the 15-25 nucleotides, e.g., 20 nucleotides, of the CD38 gene, are disposed immediately 5′ to a protospacer adjacent motif (PAM) sequence recognized by the Cas protein of the CRISPR/Cas system (e.g., where the system comprises a S. pyogenes Cas9 protein, the PAM sequence comprises NGG, where N can be any of A, T, G or C). In some embodiments, foreign DNA (e.g., DNA encoding the CAR and CCR) can be introduced into the cell along with the CRISPR/Cas system. In some embodiments, the contacting of the cells with the endonuclease system is done ex vivo. In some embodiments, the contacting is done prior to, simultaneously with, or after said cells are modified to express both CAR and CCR.

### Methods of Therapy:

A further object of the present invention relates to a method of therapy in a subject in need thereof comprising administering to the subject a therapeutically effective amount of the host immune cells of the present invention.

In particular, the host immune cells of the present invention are particularly suitable for the treatment of cancer and more particularly for the treatment of CD38-positive hematological malignancies. More particularly, the host immune cells of the present invention are particularly suitable for the treatment of multiple myeloma and even more particularly for the treatment of multiple myeloma wherein the expression of B-cell maturation antigen (BCMA) is low.

In particular, the population of host immune cells prepared as described above can be utilized in methods and compositions for adoptive immunotherapy in accordance with known techniques, or variations thereof that will be apparent to those skilled in the art based on the instant disclosure. See, e.g., US Patent Application Publication No. 2003/0170238 to Gruenberg et al; see also U.S. Pat. No. 4,690,915 to Rosenberg. Currently, most adoptive immunotherapies are autolymphocyte therapies (ALT) directed to treatments using the patient's own immune cells. These therapies involve processing the patient's own lymphocytes.

Typically, the treatments are accomplished by removing the patient's lymphocytes and transforming said cells in the population of DCAR-T cells as above described. Once the DCAR-T cells are prepared with the DCAR of the present invention, these ex vivo cells are reinfused into the patient to enhance the immune system to kill tumor calls. In some embodiments, the cells are formulated by first harvesting them from their culture medium, and then washing and concentrating the cells in a medium and container system suitable for administration (a “pharmaceutically acceptable” carrier) in a treatment-effective amount. Suitable infusion medium can be any isotonic medium formulation, typically normal saline, Normosol R (Abbott) or Plasma-Lyte A (Baxter), but also 5% dextrose in water or Ringer's lactate can be utilized.

The infusion medium can be supplemented with human serum albumin. A treatment-effective amount of cells in the composition is dependent on the relative representation of the host immune cells with the desired specificity, on the age and weight of the recipient, on the severity of the targeted condition and on the immunogenicity of the targeted Ags. These amount of cells can be as low as approximately 10³/kg, preferably 5×10³/kg; and as high as 10⁷/kg, preferably 10⁸/kg. The number of cells will depend upon the ultimate use for which the composition is intended, as will the type of cells included therein. For example, if cells that are specific for a particular Ag are desired, then the population will contain greater than 70%, generally greater than 80%, 85% and 90-95% of such cells. For uses provided herein, the cells are generally in a volume of a liter or less, can be 500 ml or less, even 250 ml or 100 ml or less. The clinically relevant number of immune cells can be apportioned into multiple infusions that cumulatively equal or exceed the desired total amount of cells.

In some embodiments, the host immune cells are formulated by first harvesting them from their culture medium, and then washing and concentrating the cells in a medium and container system suitable for administration (a pharmaceutically acceptable carrier) in a treatment-effective amount.

Thus, a further object of the present invention relates to a pharmaceutical composition comprising a population of host immune cells of the present invention and a pharmaceutically acceptable carrier.

Suitable infusion medium can be any isotonic medium formulation, typically normal saline, Normosol R (Abbott) or Plasma-Lyte A (Baxter), but also 5% dextrose in water or Ringer's lactate can be utilized. The infusion medium can be supplemented with human serum albumin. A treatment-effective amount of cells in the composition is dependent on the relative representation of the T cells with the desired specificity, on the age and weight of the recipient, on the severity of the targeted condition and on the immunogenicity of the targeted Ags. These amount of cells can be as low as approximately 10³/kg, preferably 5×10³/kg; and as high as 10⁷/kg, preferably 10⁸/kg. The number of cells will depend upon the ultimate use for which the composition is intended, as will the type of cells included therein. For example, if cells that are specific for a particular Ag are desired, then the population will contain greater than 70%, generally greater than 80%, 85% and 90-95% of such cells. For uses provided herein, the cells are generally in a volume of a liter or less, can be 500 ml or less, even 250 ml or 100 ml or less.

The clinically relevant number of immune cells can be apportioned into multiple infusions that cumulatively equal or exceed the desired total amount of cells.

The invention will be further illustrated by the following figures and examples. However, these examples and figures should not be interpreted in any way as limiting the scope of the present invention.

### FIGURES

FIG. 1: CAR T constructs and expression.
(a) Schematic diagram of the CAR construct. First generation anti-CD38 CAR (1G) is comprised of a scFv specific for CD38(Fayon et al., 2021), linked to the human CD8 hinge and transmembrane regions, followed by the human CD3 zeta intracellular signaling domain. The second generation anti-CD38 CAR (2G) is comprised of the anti-CD38 scFv linked to the human transmembrane and co-stimulation domain of CD28 and the activation domain from CD3 zeta. The DCAR was designed as a bicistronic construct. The CS1-specific scFv (Chu et al., 2014) is cloned up stream of the CD8 hinge and transmembrane regions and fused to the 4-1BB signaling domain to form the CS1 co-stimulation Receptor (CCR). The anti-CS1 CCR and 1G anti-CD38 segments are linked by a P2A self-cleaving peptide sequence. (b) Surface expression of first (1G) and second generation (2G) anti-CD38 CAR as well as DCAR determined by staining with human CD38 peptide (pCD38). (c) Histograms show the percentages of pCD38 positive cells (Left) and the Mean Intensity of fluorescence (MIF) of T cells transduced with first (1G), second generation (2G) anti-CD38 CAR, DCAR or un-transduced control (NT). Significance was calculated using an ordinary 1-way anova with Turkey's multiple comparison test. (d) FACS analysis of expression of the CD38 and the CS1 scFv on T cells transduced with the DCAR constructs using pCD38 and human CS1 peptides (pCS1). The dot plots show results from a representative donor. *p≤0.05, **p≤0.01, ***p≤0.001 ****≤0.0001

FIG. 2: Inactivation of CD38 increases the cytotoxic activities of first generation anti-CD38 CAR-T and DCAR-T cells
(a) FACS analysis of CD38 expression on T cells transduced with anti-CD38 CAR constructs. The histogram (left) shows the levels of CD38 expression on not transduced T cells (NT), or T cells transduced with first generation anti-CD38 CAR (1G), second generation anti-CD38 CAR (2G) or DCAR from one healthy donor. The bar graphs (right) show the mean±SEM of CD38 expression on NT, 1G, 2G and DCAR from 3 separate healthy donors. Significance was calculated using an ordinary 1-way anova with Turkey's multiple comparison test. (b) FACS analysis of CD38 surface expression following CRISPR/Cas9 inactivation of the CD38 gene in T cells transduced (or not) with CAR constructs as in (a). The histogram (left) shows a representative healthy donor, the bars graphs (right) show the mean±SEM of 3 separate healthy donors. Significance was calculated using an unpaired two-tailed t-test. (c-d) Surface detection of CAR molecules using a peptides CD38 showing the percentage of pCD38 positive cells (Left) and the Mean Intensity of fluorescence (MIF) bar graph or co-expression of the 1G and CCR CAR in the DCAR (d) using pCD38 and pCS1. The graphs represent the mean±SEM of 3 separate healthy donors. Significance was determined using an ordinary 1-way anova with Turkey's multiple comparison test (c) or a 2-way anova (DCAR) with Sidak's multiple comparison test (d). (e) CAR T cytotoxicity was determined by co-culturing MM.1Sluc cells for 4 hours in the presence of 1G, 2G or DCAR CAR-T cells at different effector:target (E:T) ratios. Cell viability was assessed by luciferase activity and normalized to un-Transduced T cells. Each E:T ratio shows the mean±SEM from triplicate experiments. Significance was determined by a 2-way anova with Sidak's multiple comparison test. ns≥0.05 *p≤0.05, **p≤0.01, ***p≤0.001 ****≤0.0001

FIG. 3: DCAR efficiency In vitro compared to “classic” CD38 targeting CAR-T cells. (a) CAR-T cytotoxicity was determined by co-culturing different myeloma cell lines (MM.1Sluc, KMS-11luc & HCl-H929luc) for 24 hours in the presence of 1G, 2G or DCAR CAR-T cells at different effector:target (E:T) ratios. Cell viability was assessed by luciferase activity and normalized to un-transduced-T cells (NT). Each E:T ratio shows the mean±SEM from triplicate experiments. Significance was determined by a 2-way anova with Turkeys's multiple comparison test, (b) CAR-T cell proliferation capacity was determined by a 14-day co-culture on NIH3T3 cells expressing CD38 and CS1(NIH^{38CS1}). 25.000 CAR-T cells were seeded on 100.000 NIH cells at day 0, and passed every 2-3 days on a new feeder layer. Cell number was determined using an automated cell counter. The graph represents the mean±SEM from triplicate experiments, significance was determined using a 2-way anova with Turkey's multiple comparison test. Significance is indicated for the proliferation at day 14. (c) Cytokine secretion was analyzed by taking supernatant from the CAR-T cells co-cultured on NIH3T3^{38CS1} for 4 days. Cytokine levels of interleukin (IL)-2 and tumor necrosis factor-alpha (TNF-α) were determined by a bead-based immunoassay. The graphs represent the mean±SEM from four separate experiments. Significance was determined using Mann-Whitney tests. *p≤0.05, ***p≤0.001 ****≤0.0001

FIG. 4: DCAR-T cells need both CD38 and CS1 for an “optimal/complete” anti-tumor response
(a) CAR T cytotoxicity was determined by co-culturing differentially edited MM.1Sluc cells (wt, CD38^{ko}, CS1^{ko} and double^{ko}) for 24 hours in the presence of DCAR-T cells at different effector:target (E:T) ratios. Cell viability was assessed by luciferase activity and normalized to un-transduced-T cells. Each E:T ratio shows the mean±SEM from triplicate experiments Significance was determined by a 2-way anova with Sidak's multiple comparison test. The table shows the statistical analysis for E:T ratios 0.5 and 0.1. (b) CAR-T cell proliferation capacity was determined by a 14-day co-culture on NIH3T3 cells expressing differentially edited to express either no target (NIH^{wt}), one of the targets (NIH³⁸ and NIH^{CS1}) or both targets (NIH^{38CS1}). 25.000 CAR-T cells were seeded on 100.000 NIH cells at day 0, and passed every 2-3 days on a new feeder layer. Cell number was determined using an automated cell counter. The graph represents the mean±SEM from triplicate experiments, significance was determined using a 2-way anova with Turkey's multiple comparison test. Significance is indicated for the proliferation at day 14. (c) Cytokine secretion was analyzed by taking supernatant from the CAR-T cells co-cultured on the different NIH3T3 feeder cell lines for 4 days. Cytokine levels of interleukin (IL)-2 and tumour necrosis factor-alpha (TNF-α) were determined by a bead-based immunoassay. The graphs represent the mean±SEM from four separate experiments. Significance was determined using Mann-Whitney tests. *p≤0.05, ***p≤0.001 ****≤0.0001

FIG. 5: DCAR T cells exhibit similar In Vivo activity compared to CD38 2G CAR T cells (a) Schematic representation: mice were injected intravenously with 3×10⁶ MM.1Sluc cells and 14 days later treated with 10⁶ 1G and 2G anti CD38 CAR-T cells and DCAR, a control group of un-treated (NT) mice was included in the study (n=6 for each group) (b) Representative bioluminescent images of MM.1s tumor burden from 2 independent experiments. (c) Graphical representation of tumor burden indicating the relative flux (Photons/s/cm\hat{ }2/sr) of the dorsal and ventral acquisitions combined. The bold curves represent the average radiances for each cohort, the vertical dashed line indicates the administration of CAR-T cells, while the horizontal dotted line indicates the average bioluminescence at day 35 for NT mice. (d) The survival of the mice indicated by the Kaplan-Meier method. Significance was determined using the Log-rank (Mantel-Cox) test with Bonferroni correction for multiple comparison, **p≤0.01, ***p≤0.001

FIG. 6: DCAR T cells need both targets for optimal anti-tumour response In Vivo
(a) Schematic representation: mice were injected subcutaneously with 0.5×10⁶ unmodified MM.1Sluc cells (MM.1/S^{wt}, left flank) and 0.5×10⁶ MM.1Sluc cells modified for CS1 (MM.1S^{CS1ko},right flank) cells and 4 days later treated with 10⁶ 1G and 2G anti CD38 CAR-T cells and DCAR, a control group of un-treated (NT) mice was included in the study (n=4 for each group). (b) Representative bioluminescent images of MM.1s tumor burden from 2 independent experiments. (c) Graphical representation of tumor burden indicating the total flux (Photons/s) of the dorsal acquisitions, showing MM.1S^{wt} on the left and MM.1^{CS1ko} on the right. The vertical dashed line indicates the administration of CAR-T cells.

FIG. 7: DCAR-T cells do not show hemacytotoxicity in humanized mice
(a) Schematic representation: Mice were inoculated with 0.35×10⁶ CD34+ stem cells, 39 days later mice were injected with 10⁶ CAR-T cells, Mock (MK, n=3), 2G (n=4) and DCAR (n=4). Blood samples were collected at D38, D42 and the mice were sacrificed at D46 and samples were taken for further analysis. (b) Prior to injection of CAR-T cells blood samples were taken to determine the reconstitution, the graph bar (±SEM of 11mice) indicates the percentage of the different immune populations in the CD45+ fraction. (c) The presence of circulating human cells following CAR T injection were determined by flowcytometry, the graphs indicate the mean±SEM for CD45+(Left) and CD19+(Right) cells. Significance was determined using a 2-way anova with Turkey's multiple comparison test and significance is indicated for Day 7 post CAR-T injection. (d) The weight of the mice within each treated group indicated by bar graphs ±SEM and statistical significance was determined using a 1-way anova with Turkey's multiple comparison test. (e) Interleukin (IL)-2, IL-6 and interferon gamma (IFN-γ) secretion was determined by a bead-based immunoassay on Serum samples collected on day 46. The graphs represent the mean±SEM (Mock n=3, 2G and DCAR n=4) and significance was determined using an unpaired T-test. (f) Cell count of B cells or CD38 positive (g) B cells and haematopoietic stem cells found in bone marrow samples, collected on day 46, obtained by flowcytometry. The Graphs represent the graphs indicate the mean±SEM and significance was determined using an unpaired T-test. *p≤0.05 **p≤0.01, ***p≤0.001.

### EXAMPLE

### Methods

### Construction of CAR Lentiviral Vectors

DNA sequences encoding for the anti-CD38 CAR (1G and 2G) and the anti-CS1 CCR were chemically synthetized (GeneArt ThermoFisher). Linkage of 1G anti-CD38 CAR to the P2A motif and the anti-CS1 CCR was performed using In-Fusion® snap assembly (Takara Bio) according to the manufacturers protocol. The pSFFV-Kana lentiviral vector was generated by replacing the ampicillin resistance gene of the pRRL-PGK-WPRE (a gift from Dr Hana Raslova, Villejuif, France) with the chemically synthetized Kanamycin resistance gene and by inserting the SFFV promoter in an EcoRV site, upstream of the PGK promoter. Insertion of the CAR and CCR coding sequences into the pSFFV-Kana vector was performed using In-Fusion® snap assembly.

### Cell Lines

The MM1. S, KMS11 and NCI-H929 MM cell lines have been previously described (Fayon et al., 2021). To generate Luciferase expressing cells, MM.1S, KMS11 or NCI-H929 were transduced with either lentiviral particles produced from the pLenti CMV Puro LUC (Addgene #17477) or retroviral particles. MM1. S inactivated for the CD38 and/or SLAMF7 (CS1) genes have been previously described (Fayon et al., 2021). All MM cell lines were cultured in complete RPMI medium: RPMI-1640 supplemented with 10% heat-inactivated foetal bovine serum, 100 U/ml penicillin, 100 μg/ml streptomycin and 2 nM glutamine (all from ThermoFisher) at 37 °C+5% CO2.

NIH3T3 cells were transduced with retroviral particles to obtain NIH3T3 feeder cells expressing CD38, CS1 or both targets to generate feeder cell lines. NIH3T3 cells were cultured in DMEM medium: DMEM (manufacturer) supplemented with 10% heat-inactivated FBS, 100 U/ml penicillin, 100 μg/ml streptomycin and 2 nM glutamine at 37 °C+5% CO2. HEK293-T cells were cultured under the same conditions as NH3T3 cells.

### Lentiviral Production

HEK293T cells were co-transfected with lentiviral CAR vector and packaging plasmids (pMD2G and psPAX2, all from Addgene) using calcium phosphate precipitation method (ThermoFisher) following the manufacturer's protocol. Lentiviral supernatants were collected at 72-hours post-transfection and concentrated using high-speed ultracentrifugation. To generate the lentiviral stocks, the resulting concentrated lentivirus batches were suspended in PBS and stored at −80 °C.

### CAR-T Cell Production and Genome Editing

Primary peripheral blood cells (PBMCs) were obtained from the “Etablissement Français du Sang”. In all cases, informed consent of volunteers were obtained in accordance with the Declaration of Helsinki and with approval of the Saint-Louis Hospital Internal Review Board. PBMCs were isolated from cytapheretic residues by centrifugation on a Pancoll (Human; PAN™ Biotech) gradient (2:1 ratio of diluted blood to Pancoll), using Leucosep tubes (ThermoFisher) according to the manufacturers protocol, and frozen at −80 °C in RPMI 20% SVF medium containing 10% DMSO.

PBMCs were activated with CD3/CD28 activation beads (ThermoFisher) in CTS™ OpTmizer™ T Cell Expansion SFM (Gibco) containing 100 U/ml penicillin, 100 μg/ml streptomycin, 2 nM glutamine, IL-2 (10 ng/ml, BioLegend) and hIL-7 (1 ng/μl, BioLegend), further indicated at OpTmizerC, at 37 °C+5% CO2 for 24 hours, followed by genome editing by CRISPR/Cas9. In brief, ribo-nucleoprotein (RNP) complexes were generated by incubating single guide RNA (sgRNA) targeting CD38 (100 μM, Sigma-Aldrich) was complexed with the Cas9nuclease (ThermoFisher) at a 1:2 molecular ratios, respectively, for 20 minutes at room-temperature. The RNP complex was then electroporated with 5×10⁶ activated T cells in 100 μL of 2M electroporation buffer (Chicaybam et al., 2013) in a 2B-Nucleofactor Unit (Lonza). Transfected T cells were maintained in culture, using CD3/CD28 dynabeads in OpTmizer (containing 5 ng/ml of IL-2 instead of 10 ng/ml; indicated form here as normal conditions ) at 37 °C+5% CO2, for another 24 hours prior to transduction with lentiviral particles. Lentiviral transduction was performed in culture plates coated with retronectin (15 mg/ml, Takara Bio), T cells were combined with lentiviral particles at MOI10 or higher at 37 °C+5% CO2. After 24 hours the medium was changed and cells were kept in culture under standard conditions.

### FACS Staining

For the detection of CAR expression at the surface of CAR-T cells, cells were stained in PBS1×+0.5% BSA with an anti-murine scFv-APC (JacksonImmunoResearch, #115-136-072, 1/200) or a biotinylated-CD38 peptide (Acro biosystems, #CD8-H82E7, 1/50) and a fluorescein isothiocyanate (FITC) conjugated-CS1 peptide (Acro biosystems, #SL7-HF2H7, 1/50) recognizing the respective scFvs. Following 1 hour incubation at 4 °C. Cells were washed and followed with second incubation with an APC-(BioLegend, #405207, 1/100) or BV421-(BioLegend, #405225, 1/50) streptavidin conjugated antibody for 30 min at 4 °C. Knock down efficiency was determined by staining cells with antibody directed against APC-Cy7−CD4 (BioLegend, #300518, 1/200), BUV395−CD8 (BD, #563795, 1/200) and BV510−CD38 (BD, #563251, 1/200) in FACS buffer (PBS1×, 2.5% FCS and 0.1% sodium azide) for 20 min at 4 °C. Cells were fixed in a 5% formalin solution (Sigma-Aldrich) in PBS prior to analysis. To determine reconstitution in the humanized mice cells were treated with Fc block (BD, #564220) for 10 min at RT, followed by staining with Zombie UV™ (Biolegend, #423107, 1/1000) according to the manufacturers protocol. Cells were stained for 20 min at 4 °C in FACS buffer with PerCP-CD45 (BioLegend, #304026, 1/100), BV421−CD3 (BioLegend, #317344, 1/200), SparkNir685−CD19 (Biolegend, #302270, 1/80), BUV737−CD56 (BD, #612766, 1/80), SparkBlue550−CD14 (Biolegend, #367148, 1/80), BUV805−CD15 (BD, #742057, 1/200), Alexa Flour 647-Cd1c (BioLegend, #331510, 1/200). Samples were collected using either the Fortessa (BD), Attune NxT (Invitrogen) or Cytek® Aurora (Cytek) flow cytometers and analysed using FlowJo^{tm}V10 (FlowJo Treestar).

### Cytotoxicity Assay

The cytotoxicity of CAR-T cells was determined by assessing tumour cell viability following a co-culture with CAR-T cells. Sixteen hours prior to the cytotoxicity assay, anti-CD3/CD28 beads were magnetically removed from the cultures, and CAR-T cells were mixed with luciferase expressing target cells at different effector to target (E/T) ratios. After a 4 hour or 24 hour co-culture, the luciferase activity was determined using Bright-Glo™ Luciferase Assay System (Promega) and bioluminescence was measured with a CLARIOstar plate reader (BMG Labtech). Cell viability was determined using the following formula: % of viable cells=RLU[E:T CAR]/RLU[E:T EV]*100%

### Long Term Proliferation Test

NIH3T3 feeder cells were treated with Mitomycin C (4 μg/ml, Sigma-Aldrich) for 1 hour at 37 °C, then washed with culture medium and plated in 24 well plates. CAR-T cells were co-cultured in feeder cells at an initial ration of 1:4 (25000:100000) CAR-T to feeder ratio. Every 2-3 days the CAR-T were transferred to fresh feeder cells. Cell proliferation was determined by counting the viable cells, death cells were excluded by trypan Blue (Invitrogen) staining, using the Countess II (Invitrogen). Cell numbers were quantified using ImageJ (ImageJ) software.

### Cytokine Production

Cytokine secretion was determined by collecting the supernatant of the long-term proliferation assay. The supernatant was collected at day 4 and stored at −20 °C. Using a bead-based immunoassay (LEGENDplex, Biolegend) and cytokine secretion was quantified according to the manufacturers protocol, the data was acquisitioned using the Attune NxT and analysed using the LEGENDplex software provided by the manufacturer.

### Intravenous MM1. S.luc Xenograft Model

Six to 12-week-old NOD/SCID/IL-2Rg null mice were inoculated with 3×10⁶ MM1. SLuc cells by tail vein injection (i.v) at day 0, followed, 14 days later, by infusion of 10⁶ CAR-T. Bioluminescence was measured with the IVIS Imaging System (PerkinElmer) every 7 days after tumour injection. The study contained 6 mice in each group which were injected with CAR-T cells generated from 3 different healthy donors. Data was further analysed using Aura imaging software (Spectral Instruments Imaging)

### Subcutaneous MM1. S.luc Xenograft Model

MM1. Sluc cells (0.5×10⁶ cells per mouse) were injected sub cutaneous on the back of NSG mice. MM1. S^{wt} cells were injected in the left flank and MM1. S^{CS1ko} cells were injected in the right flank of the mice. CAR-T cells (10⁶ cells per mouse) were administered IV in the tail vein 4 days post tumor injection. Tumor progression was followed by luminescent imaging every 7 days using the IVIS Imaging System. The study contained 4 mice in each group which were injected with CAR-T cells originating from 2 different healthy donors. Data was further analysed using Aura imaging software (Spectral Instruments Imaging).

### Humanized Mice Model

Haematopoietic progenitors were obtained from cord blood by centrifugation on a Pancoll gradient (2:1 ratio of diluted blood to Pancoll) using Leucosep tubes according to the manufacturers protocol. CD34+ cells were isolated by magnetic cell sorting using a CD34 MicroBead Kit (Miltenyi Biotec) according to the manufacturers protocol and frozen at −80 °C or cultured immediately. Fresh or frozen CD34+ cells were cultured in EIDM medium: EIDM (Gibco) completed with 20% BIT (Stemcell™ technologies), 100 U/ml penicillin, 100 μg/ml streptomycin, 2 nM glutamine, 50 ng/ml SCF (Miltenyi Biotec), 50 ng/ml FLT3 (Miltenyi Biotec) 20 ng/ml TPO (Miltenyi Biotec), 10 ng/ml IL-3 (Miltenyi Biotec) for 16H at 37 °C+5% CO2. The CD34+ cells (4 different donors) were pooled and cells (0.35×10⁶ per mouse) were administered IV in the tail vein in sub lethally irradiated NSG mice. 5-weeks post injection of CD34+ cells mice injected with CAR-T cells (10⁶ cells per mouse) IV in the tail vein. Each CAR-T was produced from 2 different healthy donors. Blood was collected from the mice to monitor the reconstitution prior to CAR-T cell injection. Blood, serum and bone marrow were collected post CAR-T cell treatment for further analysis by flow cytometry. Prior to analysis blood and organ samples were passed on a Pancoll gradient (2:1 ratio of diluted blood to Pancoll).

### Statistics

Statistical tests were performed using GraphPad Prism V9 (GraphPad), statistical tests used for each figure are indicated in the corresponding figure legends.

### Results

### Construction of Anti-CD38 and Dual Anti-CD38/CS1-CARs

To investigate novel CAR-T targeting CD38 in MM, we designed a first generation anti CD38 CAR (1G) containing a recently published anti-CD38 scFv (Fayon et al 2020) fused with the human CD8 transmembrane region (TM) and the CD3z signalling domain (FIG. 1a). We also assembled a second-generation CAR (2G) by fusing the anti-CD38 scFv to the TM and signalling domains of the CD28 co-stimulatory receptor and to the transduction region of the CD3z chain (FIG. 1a). Then, we designed a dual-CAR (DCAR) with different antigen specificities, one of which contained the sequence of the anti-CD38 1G CAR, while the other one contained the 4-1BB costimulatory domains (CCR) and targeted CS1 through the anti-CS1 scFv (Chu et al., 2014). DNA sequences encoding the anti-CD38 1G CAR and the anti-CS1 CCR were cloned into a P2A-based bicistronic construct, which allows the expression of two separate polypeptides from a single gene (FIG. 1a). All sequences coding for 1G, 2G and DCAR were cloned under the control of the spleen focus-forming virus (SFFV) promoter into a lentiviral vector and were stably expressed on T cells isolated from healthy human donors. FACS analysis of transduced T cells stained with a anti human scFv revealed strong expression of the CARs and/or CCR transgenes in around 90% of infected T cells (data not shown). Surface expression of anti-CD38 CARs was further explored by staining transduced T cells with a biotinylated CD38 peptide. We observed that T cells transduced with 1G and 2G displayed higher expression levels of anti-CD38 CAR compare with those infected with DCAR (FIGS. 1b and 1c). Importantly, staining with CD38 and CS1 peptides coupled with different dyes revealed the combined expression of anti-CD38 CAR and anti-CS1 CCR on more than 60% of DCAR transduced T cells from healthy donors (FIG. 1d). Thus, the DCAR construct allows co-expression of the anti-CD38 CAR and the anti-CS1 CCR, while 1G and 2G vectors induced a higher expression level of the anti-CD38 CAR at the surface of transduced T cells.

Deletion of CD38 in Anti-CD38 DCAR-T Enhances their Cytotoxic Activity Against MM Cells

Since CD38 is upregulated on activated T cells, we investigated whether this process could interfere with the expression or the function of anti-CD38 CAR. We first analysed expression of CD38 at the surface of anti-CD38 CAR-T (1G and 2G), and DCAR-T, derived from healthy donors, compare with not transduced T cell controls. FACS analysis revealed a marked reduction of CD38 expression on CAR- and DCAR-T compared to controls (FIG. 2a). Next, we developed CRISPR/Cas9 mediated CD38 gene inactivation to investigate whether expression of CD38 could impair anti-CD38 CAR expression at the surface of activated T cells. Transfection of Cas9 ribonucleoprotein (RNP) targeting the CD38 gene inactivated CD38 expression in around 90% of non-transduced control T cells (FIG. 2b). As expected, CD38 was almost not detected on CD38 edited CAR- and DCAR-T (data not shown). Interestingly, FACS analysis of CAR-T stained with an CD38 peptide showed that edition of the CD38 gene did not affect the expression levels of the anti-CD38 CAR at the cell surface of transduced T cells (FIG. 2c) nor the percentage of DCAR-T co-expressing the 1G CAR and the CCR (FIG. 2d). Thus, expression of the anti-CD38 CAR inhibits CD38 expression at the surface of activated T cells, however, inactivation of CD38 has no effect on the levels of anti-CD38 CAR expression.

Then, we explored whether inactivation of the CD38 gene could affect the cytotoxic activity of anti-CD38 CAR- and DCAR-T against CD38 expressing MM cells. We performed co-culture assays with firefly luciferase expressing CD38 positive MM1. S MM cell lines (MM1. Sluc) and transduced T cells, edited or not for CD38. Luciferase levels, which reflect the numbers of live MM cells, were measured after a 4 hour co-culture of effector CAR-T (E) and target MM1. Sluc (T) mixed at ratios varying from 10:1 to 1:3. We observed that, while both 1G anti-CD38 CAR and DCAR transduced not edited T cells efficiently killed MM cells in vitro, edition of the CD38 gene rendered CAR-T cells significantly more efficient to lyse MM1. S cells at most E:T ratios (FIG. 2e). Of note, edition of CD38 had no statistically significant impact on the cytotoxic activity of 2G CAR T against MM cells (FIG. 2e). These data indicate that inactivation of CD38 markedly enhanced the anti-MM cytotoxic activity of 1G anti-CD38 CAR-T and DCAR-T cells. These results prompted us to used CD38 edited T cells for the following experiments.

### DCAR-T Cells Develop Strong Responses Against Cells Expressing CD38 and CS1 Antigens

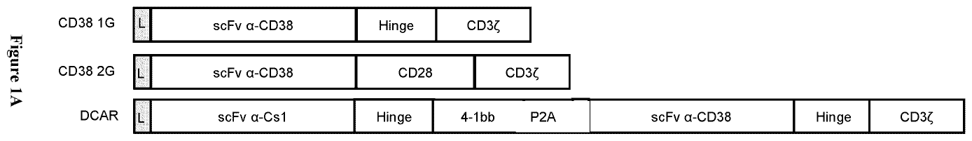

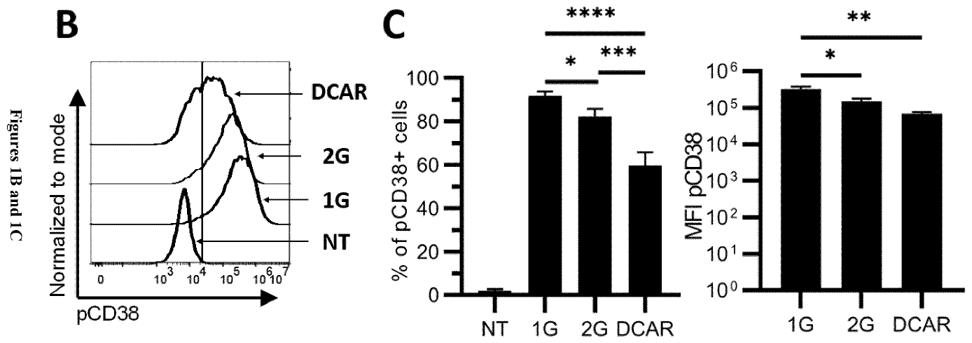

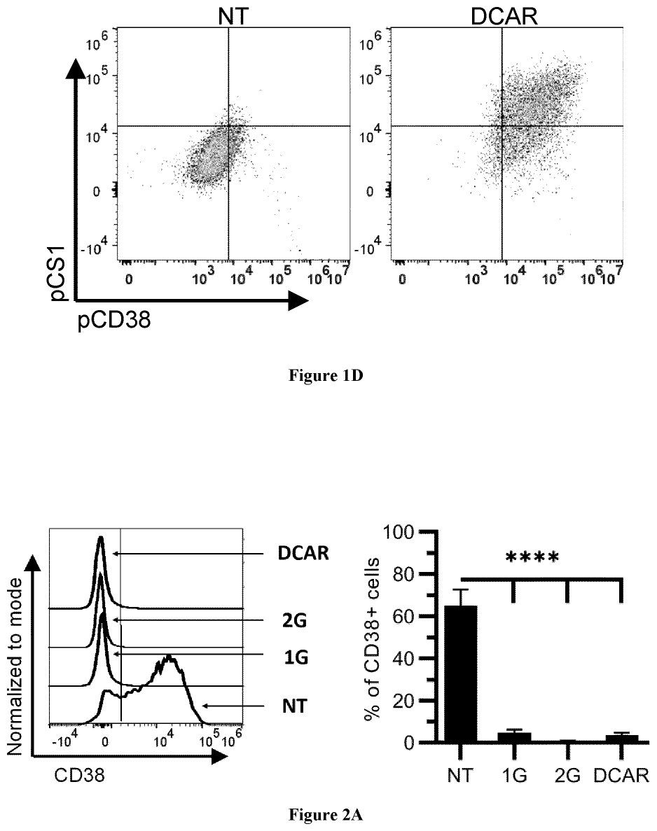

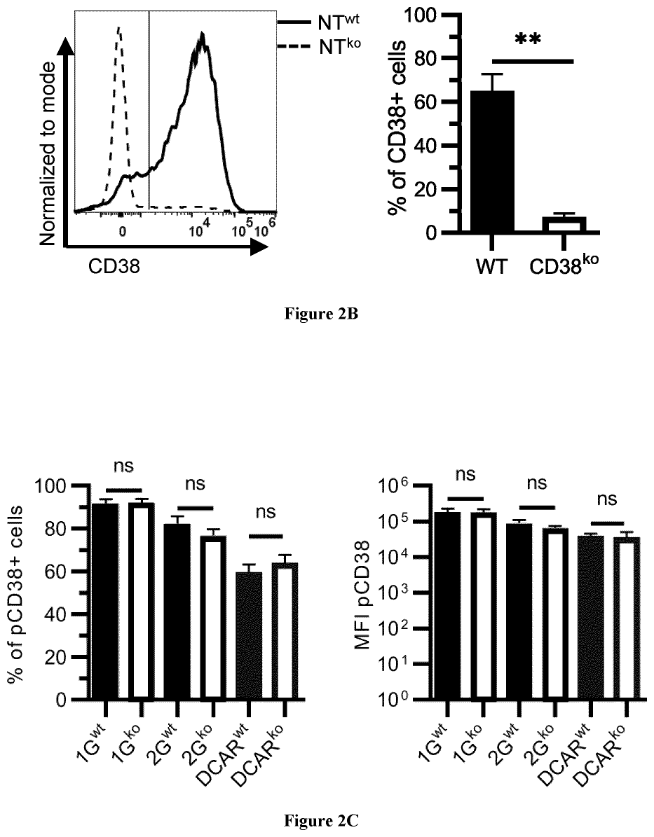

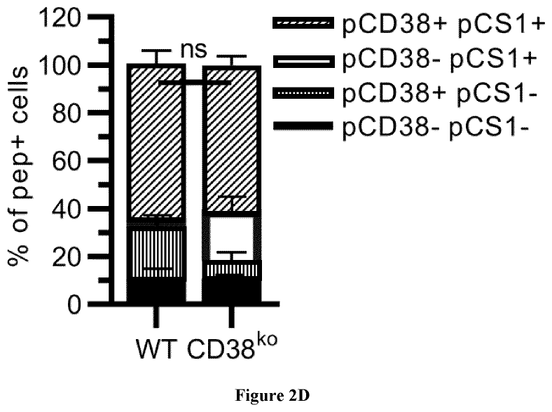

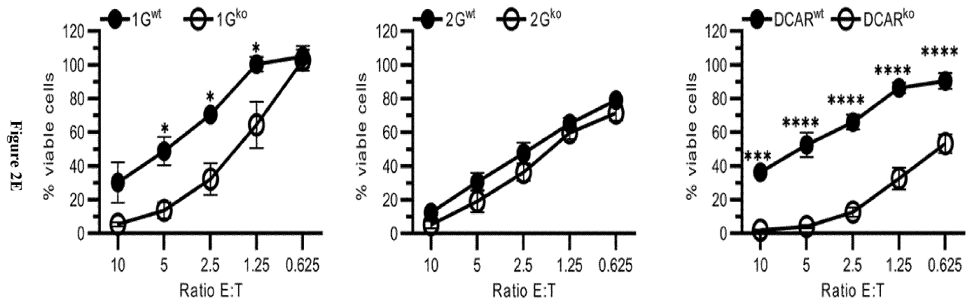

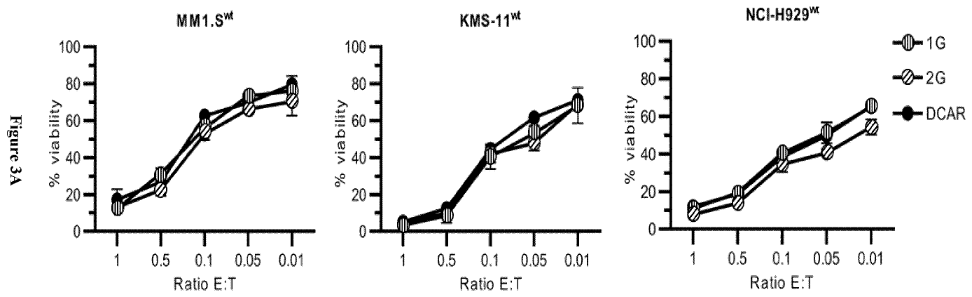

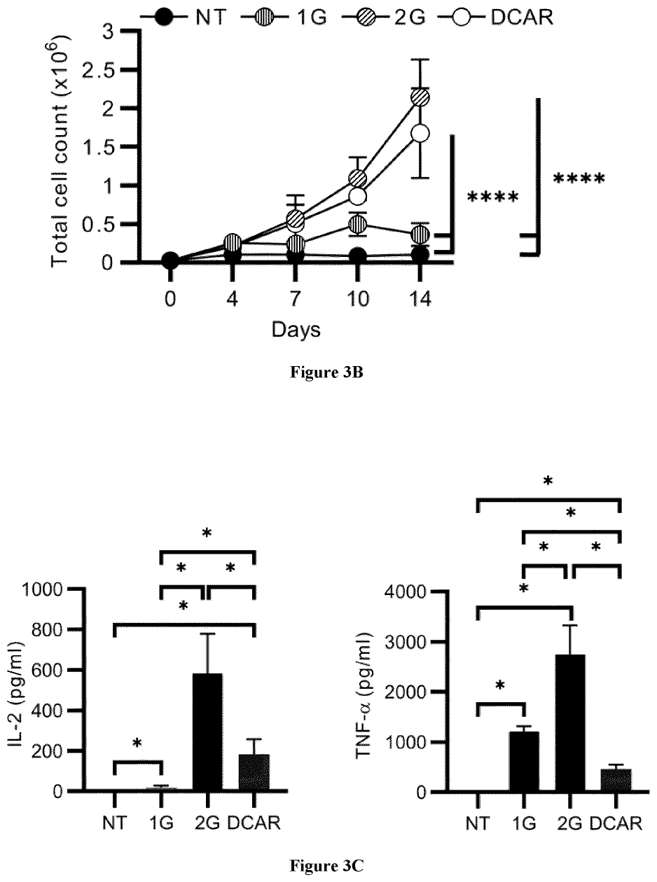

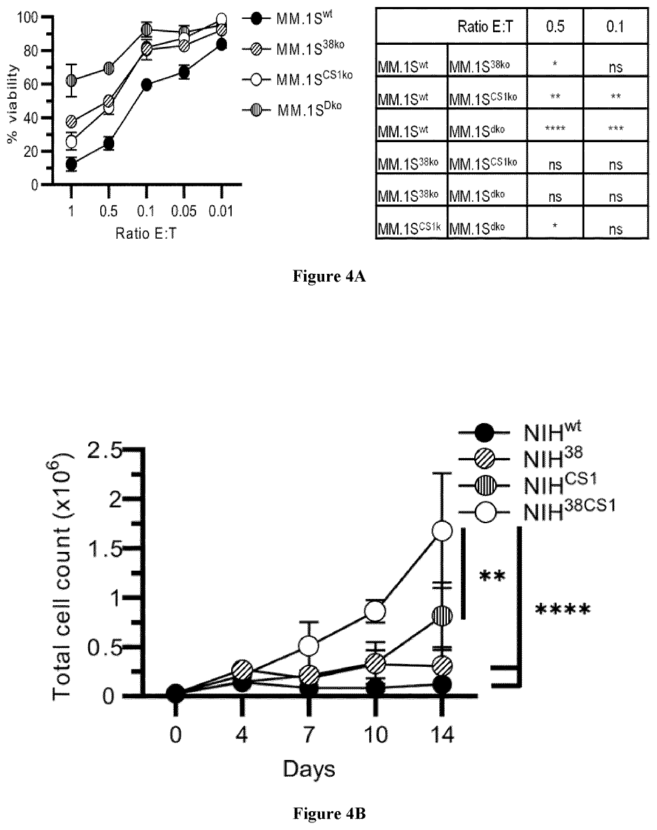

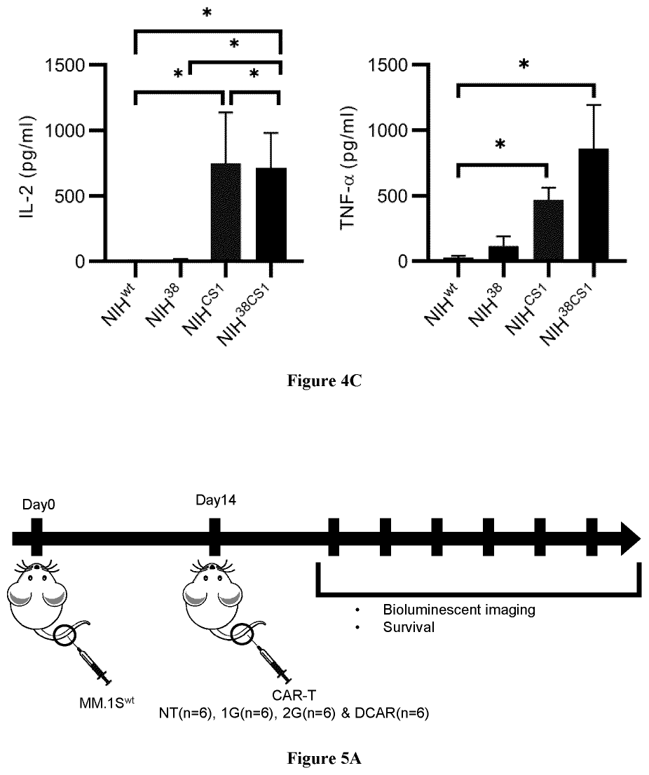

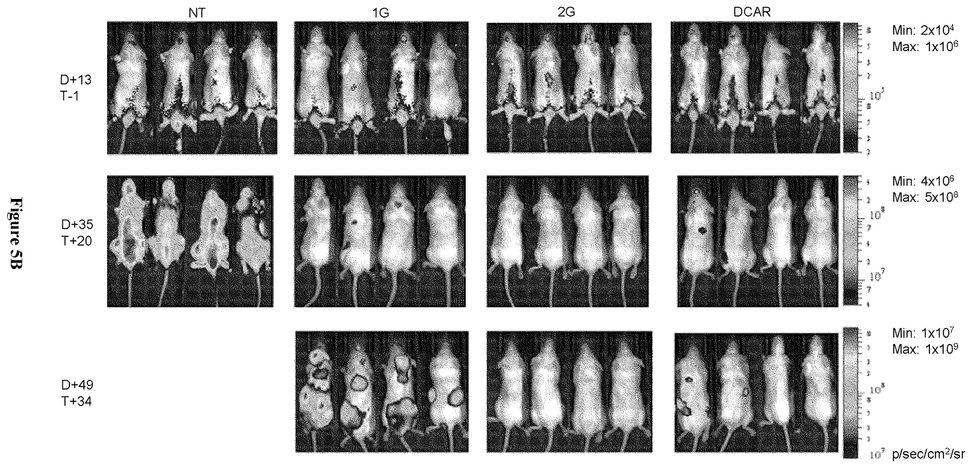

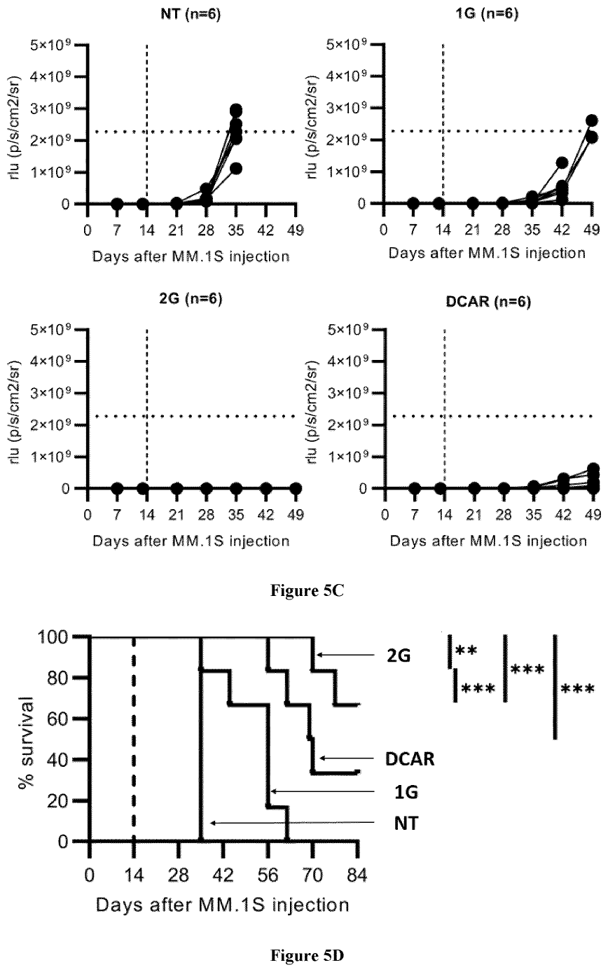

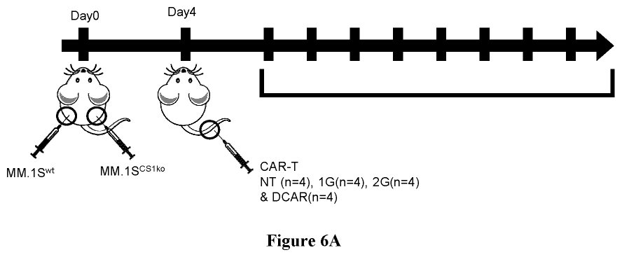

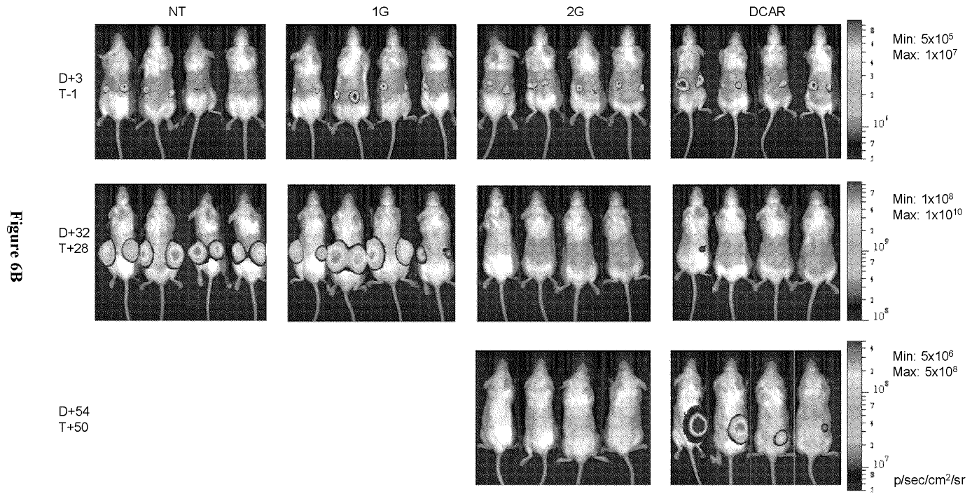

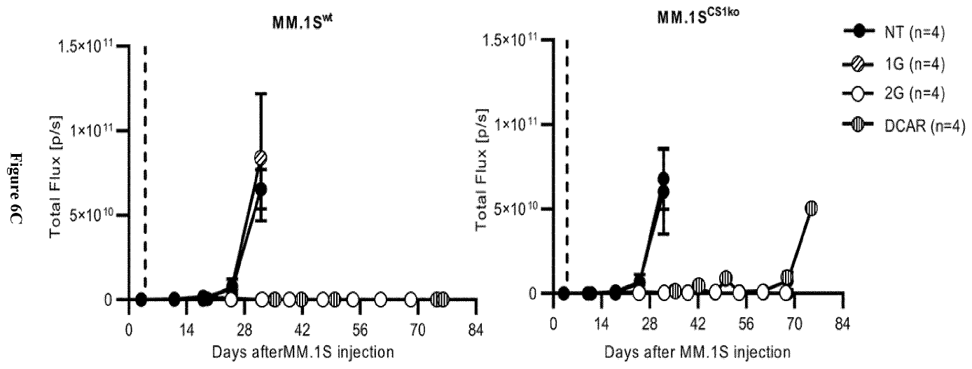

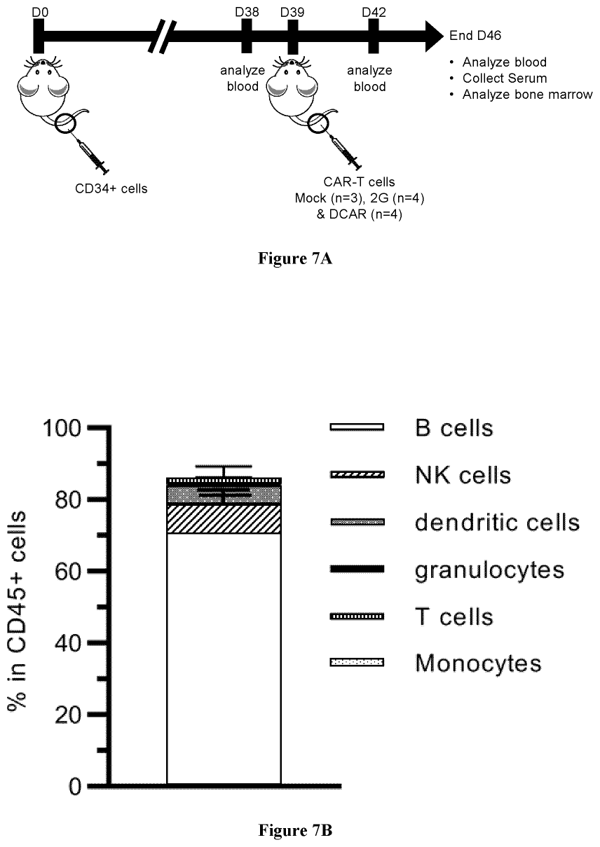

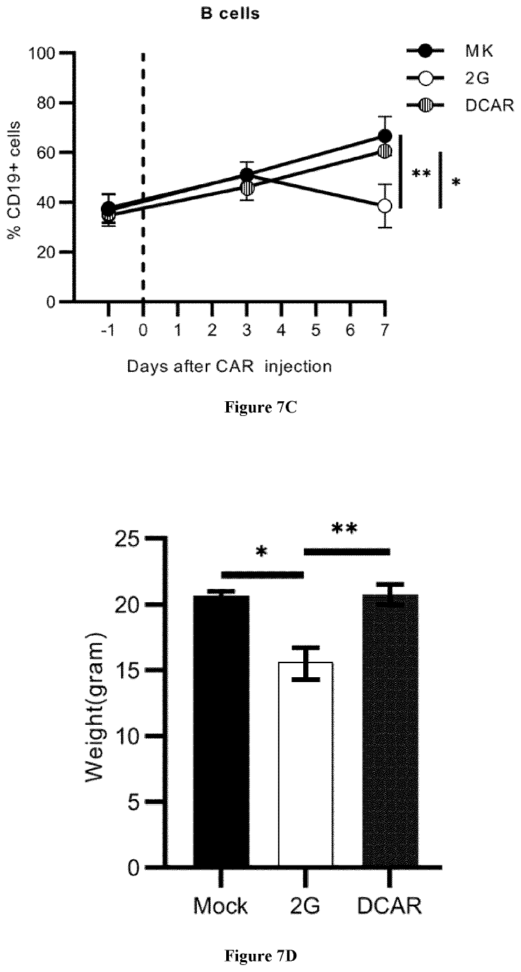

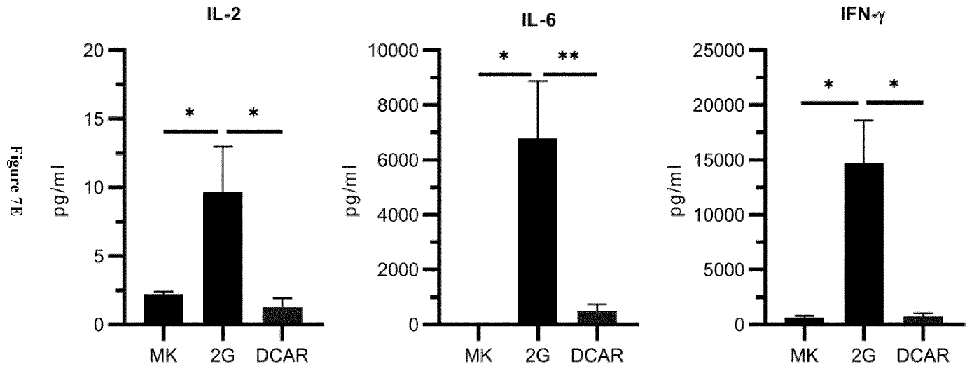

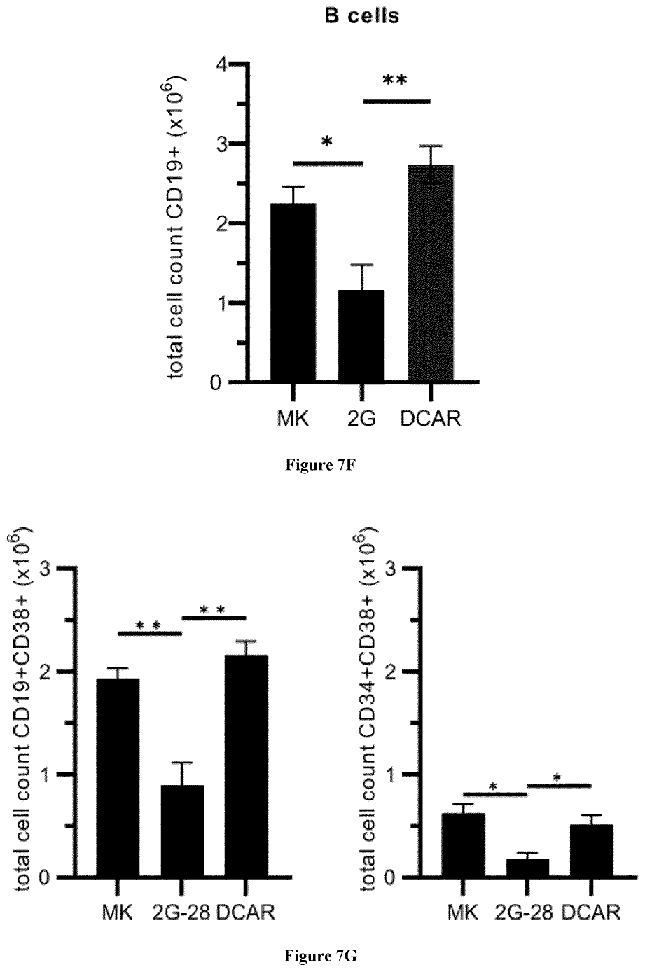

Efficiency of CAR-T cells relies on complex processes, including affinity of the chimeric receptor to its target, abundance of the recognized antigen on target cells, CAR expression levels or composition of the signalling module (Rafiq et al., 2020). Considering the various expression levels of the anti-CD38 CAR driven by our constructs and the different structures of their signalling domains, we assayed the cytotoxic activities of DCAR-T, 1G and 2G anti-CD38 CAR-T cells in vitro. CD38 edited T cells from healthy donors were transduced with the different vectors, expanded in vitro for 7-14 days with anti-CD3/CD28 beads plus interleukine-2 (IL-2) and IL-7, then co-cultured overnight with MM1. Sluc at various E:T ratios and the luciferase levels were measured to determine the cytotoxic activity. We observed similar cytotoxic activities for all CAR and DCAR T cells against MM1. S cells, with around 60% cell viability at an E:T ratio of 1:10 (FIG. 3a). We repeated the killing assays with NCI-H929luc and KMS11luc MM cell lines as targets and, again, we found that all 3 CAR constructs provided between 40 and 60% cytotoxic activities against target cells at the E:T ratio of 1:10. Thus, DCAR, 1G and 2G anti-CD38 CAR-T cells displayed strong and similar anti-MM killing activity in vitro.

Association of CD28 or 4-1BB signalling domains with the intracellular CD3z chain region has been shown to enhance both expansion and cytokine production by CAR-T in response to their cognate antigen (Maher et al., 2002). To test the proliferation of CD38 edited T cells expressing our anti-CD38 CAR and DCAR constructs, we established an in vitro assay to measure the expansion of the T cells cultured on NIH3T3 cells expressing both CD38 and CS1 (NIH^{38CS1}) Our results show that 2G anti-CD38 CAR-T and DCAR-T readily proliferated in vitro, expanding from 25000 cells at day 0 to more than 1.5×10⁶ cells at day 14 (FIG. 3b). In contrast, 1G anti-CD38 CAR-T and not transduced T cells displayed little or no proliferation over time. Furthermore, we used this NIH cells model to measure the production of IL-2 and tumour necrosis factor-alpha (TNF-α) by T cells transduced with the different constructs. After 96 hours in culture on NIH^{38CS1} production of IL-2 was much lower in 1G than in 2G anti-CD38 CAR-T cultures (18.73 pg/mL compare with 580 μg/mL) (FIG. 3c). Remarkably, DCAR-T secreted an intermediate level of IL-2 (183 pg/mL), while no production was detected in the culture medium of not transduced control T cells. Importantly, TNF-α was readily produced by all transduced T cells, however the levels were higher in 1G and 2G anti-CD38 CAR-T compared with DCAR-T (1.2 pg/mL, 2.7 pg/mL and 0.45 pg/mL, respectively as average) (FIG. 3c). These results indicate that DCAR-T responded to stimulation by CD38 and CS1 expressing targets by secreting strong levels of IL-2 and lower levels of TNF-α compare to 1G and 2G anti-CD38 CAR-T, in vitro.

### DCAR-T Require Expression of Both CD38 and CS1 on Target Cells to Deliver Efficient Responses

In order to decipher the role of each CAR and CCR receptors in the responses of DCAR-T to target cells, we developed a CRISPR/Cas9 mediated approach to inactivate the CD38 and/or SLAMF7 (encoding for CS1) genes in the MM1. Sluc cell line (Fayon et al., 2021) and used the edited cells to assay the cytotoxic activity of in vitro expanded CD38 edited DCAR-T cells. As expected, after overnight co-culture, DCAR-T efficiently killed un-manipulated MM1. Sluc cells (MM1. S^{wt}), even at low E:T ratios (20% viability at E:T of 1:2) (FIG. 4a). In contrast, MM1. S cells inactivated for both CD38 and CS1 (MM1. S^{Dko}), appeared significantly resistant to DCAR-T cell lysis as 60% of the cells remained viable at the E:T of 1:2, as average, and 90% at the E:T of 1:10 (FIG. 4a). Of note, MM cells inactivated for CD38 (MM1. S^{38ko}) or for CS1 (MM1. S^{CS1ko}) displayed intermediate sensitivities to DCAR-T mediated killing, with around 50% and 75% of viable cells at E:T of 1:2 and 1:10, respectively (FIG. 4a). To test the function of each CAR and CCR receptor in cell proliferation, we generated NIH3T3 cells expressing CD38 (NIH³⁸), CS1 (NIH^{CS1}), CD38 and CS1 (NIH^{38CS1}) or none (NIH) and used each cell line to support CD38 edited DCAR-T cell expansion in vitro. We observed that DCAR-T stimulated with either NIH^{wt} or NIH³⁸ cells expanded very poorly (FIG. 4b). In contrast, culture on NIH^{38CS1} feeder cells triggered a robust proliferation of DCAR-T, with cells numbers increasing from 25000 at day 0 to more than 1.5×10⁶ at day 14. Of note, DCAR-T co-culture on NIH^{CS1} cells readily expanded, albeit at a lower level compare to those cultured on NIH^{38CS1} cells expressing both CD38 and CS1 antigens. Next, we used the NIH3T3 cellular models to investigate in vitro cytokine production by edited DCAR-T in response to engagement of anti-CD38 CAR, anti-CS1 CCR or both. As expected, the medium of DCAR-T cultured for 96 hours on NIH^{wt} contained low concentrations of IL-2 and TNF-α (FIG. 4c). Similarly, we found that DCAR-T produced weak levels of IL-2 and TNF-α when cultured on NIH³⁸. However, both cytokines were abundantly produced following culture on NIH^{CS1} or NIH^{38CS1} cells (FIG. 4c). Altogether, these results indicate that combined expression of CD38 and CS1 antigens on target cells is required for DCAR-T to efficiently kill MM cells, to proliferate robustly and to produce high levels of IL-2 in vitro.

### DCAR-T Provide Long Term Control of MM Tumor Progression In Vivo.

We next tested the efficacy of the different CAR constructs to direct T-cell anti myeloma activity in vivo. We established a xenograft mouse model by inoculating MM1. Sluc cells intra-venously into NOD/SCID/γc^{-/-} (NSG) immune-deficient mice and followed luciferase levels with an IVIS Imaging System. Fourteen days after MM1. S injection, non-transduced control T cells, anti-CD38 CAR-T (1G or 2G) or DCAR-T were transplanted into engrafted mice and bioluminescence measured every 7 days (FIG. 5a). Tumor burden increased rapidly in mice that received no treatment (NT) with an average luminescence of 2.3×10⁹ p/s/cm2/sr at day 35 post transplantation (FIG. 5b). In contrast, injection of 2G anti-CD38 CAR-T readily controlled tumor growth over time (FIG. 5b). As expected, treatment with 1G anti-CD38 CAR-T was less efficient to control MM progression long term, with the average luminescence levels reaching 2.4×10⁹ p/s/cm2/sr at day 49. Strikingly, mice that received DCAR-T cells showed prolonged control of tumor growth compared with mice treated with 1G anti-CD38 CAR-T (FIG. 5c). In line with this, we observed that mice injected with CD38 2G and DCAR had a significant elongation of survival compared to NT and 1G treated mice (FIG. 5d). Thus, DCAR-T as 2G anti-CD38 CAR-T induced efficient and long-term inhibition of MM tumor progression in vivo.

### DCAR-T Display Low Cytotoxicity Against CS1 Deficient MM Cells In Vivo

One major pit fall of anti-CD38 CAR-T approach is their potential off tumor activity, which could lead to sever side effects. In order to explore the specificity of the DCAR system to targets expressing both CD38 and CS1 antigens, we developed and an in vivo model by injecting MM1. S^{t} and MM1. S^{CS1ko} cells in the right and left flanks of mice, respectively, and assay for the ability of each CD38 edited anti-CD38 CAR-T to inhibit tumor growth (FIG. 6a). Eight days after subcutaneous injection of MM1. S^{t} and MM1. S^{CS1ko} cells, tumors were readily detected on both flanks of the mice (FIG. 6b). Then, tumor bearing mice were randomized and inoculated with non-transduced T cells, 1G, 2G anti-CD38 CAR-T or with DCAR-T and bioluminescence was monitored by IVIS over time. Expansion of MM1. S′ and MM1. S^{CS1ko} cells was observed in mice treated with control T cells and 1G anti-CD38 CAR-T and by day 32, the tumor burden on each flank has markedly increased (FIG. 6b). In contrast, treatment with 2G anti-CD38 CAR-T eradicated both MM1. S′ and MM1. S^{CS1ko} tumours and no sign of progression could be observed during the time of the experiment. Remarkably, injection of DCAR-T efficiently eliminated MM1. S′ cells, however, in the same mouse, by day 70 expansion of MM1. S^{CS1ko} cells readily occurred (FIGS. 6b and c). Thus, DCAR-T cells selectively control the tumor progression of cells bearing both CD38 and CS1, while preserving CD38 targets lacking expression of CS1 in vivo.

### DCAR-T Cells Preserve Haematopoietic Cells in Humanized Mice

To further investigate potential toxicities of anti-CD38 CAR-T in vivo, we generated humanized mice by injecting CD34 positive human hematopoietic stem cells (HSC) from cord blood into NSG recipient mice (Alhaj Hussen et al., 2017), and used this model to assay for anti-CD38 CAR- and DCAR-T haemato-toxicity (FIG. 7a). Five weeks post HSC injection, blood cells from each mice were collected and analysed to verify the development of human haematopoiesis. We found similar reconstitution of human haematopoietic cells in all mice, with the great majority of CD45 expressing cells being B lineage cells staining for CD19 (FIG. 7b). Mice were then randomized into 3 cohorts and injected with non-transduced T cells, 2G anti-CD38 CAR-T or DCAR-T, all inactivated for CD38. Blood samples were collected 1 day before CAR-T inoculation, 3 days after treatment as well as at day 7, when mice were sacrificed and analysed by flow cytometry. While the percentages of CD19+B lineage cells were similar in all mice before CAR-T injection and at day 3 post treatment, these percentages were markedly reduced at day 7 in mice inoculated with 2G anti-CD38 CAR-T compare to DCAR and control T cells (FIG. 7c). In the meantime, we observed that the mice treated with 2G CAR-T had lost about 10% of their weight compare to DCAR-T or control T cells (FIG. 7d). Analysis of the serum revealed a marked elevation of the levels of human IL-2, IL-6 and INF-γ compared with animal injected with DCAR-T or control T cells (FIG. 7e). To further explore the toxicity of anti-CD38 CAR-T in vivo, we analysed the bone marrow cell populations of mice in each cohort. In line with the results in blood, we observed a significant reduction in the numbers of human CD19+B lineage cells in mice that were injected with 2G anti-CD38 CAR-T compared with those that received DCAR-T or control T cells (FIG. 7f). Since in this mouse model, more than 85% of B and HSC express CD38 (data not shown), there was a similar reduction of CD38 expressing CD19+ and CD34+ cells in mice treated with 2G-28 CAR-T (FIG. 7g). Altogether, these results suggest that injection of 2G anti-CD38 CAR-T into humanized mice impairs the haematopoietic cell populations of the blood and the bone marrow and triggers a strong immune reaction as measured by cytokine secretion. Conversely, inoculation of DCAR-T has no detectable toxicity towards human haematopoietic cells in vivo.

### CONCLUSIONS

Here we present DCAR, a split double CAR setting targeting MM tumour cells via the recognition of CD38 by an activation receptor bearing the CD3z signalling domain and CS1 through a co-stimulation receptor containing the 4-1BB intracellular domain. Expression of the DCAR anti-CD38 and anti-CS1 activation and co-stimulation chimeric antigen receptors is driven by an original kanamycin resistant lentiviral vector which can be approved for clinical assays. We provide evidence that inactivation of the CD38 gene by CRISPR/Cas9 edition increases DCAR-T cells killing of MM cells in vitro. Key features of DCAR include robust killing of MM cells in vitro and in vivo; high levels of IL-2 production in response to targets expressing both CD38 and CS1; and, as safety feature, the lack of detectable haemato-toxicity in vivo. Thus, combine inactivation of CD38 and DCAR expression in T cells could represents a promising therapeutic approach for MM.

### REFERENCES

Throughout this application, various references describe the state of the art to which this invention pertains. The disclosures of these references are hereby incorporated by reference into the present disclosure.
Alhaj Hussen, K., Vu Manh, T. P., Guimiot, F., Nelson, E., Chabaane, E., Delord, M., Barbier, M., Berthault, C., Dulphy, N., Alberdi, A. J., et al. (2017). Molecular and Functional Characterization of Lymphoid Progenitor Subsets Reveals a Bipartite Architecture of Human Lymphopoiesis. Immunity 47, 680-696 e688.
Boyiadzis, M. M., Dhodapkar, M. V., Brentjens, R. J., Kochenderfer, J. N., Neelapu, S. S., Maus, M. V., Porter, D. L., Maloney, D. G., Grupp, S. A., Mackall, C. L., et al. (2018). Chimeric antigen receptor (CAR) T therapies for the treatment of hematologic malignancies: clinical perspective and significance. J Immunother Cancer 6, 137.
Chicaybam, L., Sodre, A. L., Curzio, B. A., and Bonamino, M. H. (2013). An efficient low cost method for gene transfer to T lymphocytes. PLoS One 8, e60298.
Chu, J., Deng, Y., Benson, D. M., He, S., Hughes, T., Zhang, J., Peng, Y., Mao, H., Yi, L., Ghoshal, K., et al. (2014). CS1-specific chimeric antigen receptor (CAR)-engineered natural killer cells enhance in vitro and in vivo antitumor activity against human multiple myeloma. Leukemia 28, 917-927.
Da Via, M. C., Dietrich, O., Truger, M., Arampatzi, P., Duell, J., Heidemeier, A., Zhou, X., Danhof, S., Kraus, S., Chatterjee, M., et al. (2021). Homozygous BCMA gene deletion in response to anti-BCMA CAR T cells in a patient with multiple myeloma. Nat Med 27, 616-619.
Dimopoulos, M. A., Oriol, A., Nahi, H., San-Miguel, J., Bahlis, N.J., Usmani, S. Z., Rabin, N., Orlowski, R. Z., Komarnicki, M., Suzuki, K., et al. (2016). Daratumumab, Lenalidomide, and Dexamethasone for Multiple Myeloma. N Engl J Med 375, 1319-1331.
Dimopoulos, M. A., Richardson, P., and Lonial, S. (2022). Treatment Options for Patients With Heavily Pretreated Relapsed and Refractory Multiple Myeloma. Clin Lymphoma Myeloma Leuk.
Fayon, M., Martinez-Cingolani, C., Abecassis, A., Roders, N., Nelson, E., Choisy, C., Talbot, A., Bensussan, A., Fermand, J. P., Arnulf, B., et al. (2021). Bi38-3 is a novel CD38/CD3 bispecific T-cell engager with low toxicity for the treatment of multiple myeloma. Haematologica 106, 1193-1197.
Kloss, C. C., Condomines, M., Cartellieri, M., Bachmann, M., and Sadelain, M. (2013). Combinatorial antigen recognition with balanced signaling promotes selective tumor eradication by engineered T cells. Nat Biotechnol 31, 71-75.
Lanitis, E., Poussin, M., Klattenhoff, A. W., Song, D., Sandaltzopoulos, R., June, C. H., and Powell, D. J., Jr. (2013). Chimeric antigen receptor T Cells with dissociated signaling domains exhibit focused antitumor activity with reduced potential for toxicity in vivo. Cancer Immunol Res 1, 43-53.
Laurent, S. A., Hoffmann, F. S., Kuhn, P. H., Cheng, Q., Chu, Y., Schmidt-Supprian, M., Hauck, S. M., Schuh, E., Krumbholz, M., Rubsamen, H., et al. (2015). gamma-Secretase directly sheds the survival receptor BCMA from plasma cells. Nat Commun 6, 7333.
Maher, J., Brentjens, R. J., Gunset, G., Riviere, I., and Sadelain, M. (2002). Human T-lymphocyte cytotoxicity and proliferation directed by a single chimeric TCRzeta/CD28 receptor. Nat Biotechnol 20, 70-75.
Morgan, G. J., Walker, B. A., and Davies, F. E. (2012). The genetic architecture of multiple myeloma. Nat Rev Cancer 12, 335-348.
Munshi, N.C., Anderson, L. D., Jr., Shah, N., Madduri, D., Berdeja, J., Lonial, S., Raje, N., Lin, Y., Siegel, D., Oriol, A., et al. (2021). Idecabtagene Vicleucel in Relapsed and Refractory Multiple Myeloma. N Engl J Med 384, 705-716.
Rafiq, S., Hackett, C. S., and Brentjens, R. J. (2020). Engineering strategies to overcome the current roadblocks in CAR T cell therapy. Nat Rev Clin Oncol 17, 147-167.
Sadelain, M., Brentjens, R., and Riviere, I. (2013). The basic principles of chimeric antigen receptor design. Cancer Discov 3, 388-398.

1. A host immune cell engineered to express a) a chimeric antigen receptor (CAR) that binds to CD38, wherein binding of the CAR to CD38 delivers an activation signal to the host immune cell, and b) a chimeric co-stimulating receptor (CCR) that binds to a second antigen, wherein binding of the CCR to the second antigen delivers a costimulatory signal to the host immune cell but does not deliver an activation signal to the host immune cell, wherein the host immune cell (i) exhibits negligible cytotoxicity against cells that are single positive for CD38, and (ii) induces cytotoxicity against cells that are positive for both CD38 and the second antigen.

2. The host immune cell of claim 1 that is selected from the group consisting of a T cell, a Natural Killer (NK) cell, a cytotoxic T lymphocyte (CTL), a human embryonic stem cell, and a pluripotent stem cell from which lymphoid cells are differentiated.

3. The host immune cell of claim 1 wherein the chimeric antigen receptor (CAR) comprises an extracellular domain and an intracellular signalling domain joined by a transmembrane domain, and wherein the extracellular domain, expressed on the surface of the host immune cell, comprises an antigen binding domain having binding affinity for CD38 and the intracellular signalling domain produces an activation signal to the host immune cell upon recognition of the antigen by the extracellular domain.

4. The host immune cell of claim 3 wherein the antigen binding domain is a scFv comprising a VH domain comprising i) the H-CDR1 as set forth in SEQ ID NO:3, ii) the H-CDR2 as set forth in SEQ ID NO:4 and iii) the H-CDR3 as set forth in SEQ ID NO:5, and, a VL domain comprising i) the L-CDR1 as set forth in SEQ ID NO:6, ii) the L-CDR2 as set forth in SEQ ID NO:7 and iii) the L-CDR3 as set forth in SEQ ID NO:8.

5. The host immune cell of claim 4 wherein the scFv comprises a VH domain having at least 90% of identity with the amino acid sequence as set forth in SEQ ID NO:9 and/or a VL domain having at least 90% identity with the amino acid sequence as set forth in SEQ ID NO:10; and/or the scFv comprises an amino acid sequence having 90% of identity with the amino acid sequence as set forth in SEQ NO:11.

6. (canceled)

7. The host immune cell of claim 3 wherein the transmembrane domain comprises the amino acid sequence having at least 90% identity with the amino acid sequence as set forth in SEQ ID NO:12.

8. The host immune cell of claim 3 wherein the intracellular signalling domain is derived from an intracellular signalling domain of a lymphocyte receptor chain, a TCR/CD3 complex protein, an Fc receptor subunit, an IL-2 receptor subunit, CD3Q, FcRγ, FcRβ, CD37, CD3δ, CD3s, CD5, CD22, CD79a, CD79b, CD66d, CD278(ICOS), FcsRI, DAP10, or DAP12.

9. (canceled)

10. The host immune cell of claim 8 wherein the intracellular signalling domain in the CAR comprises an amino acid sequence having at least 90% identity with the amino acid sequence as set forth in SEQ ID NO:13; and/or the CAR comprises an amino acid sequence having at least 90% of identity with the amino acid sequence as set forth in SEQ ID NO:14.

11. (canceled)

12. The host immune cell of claim 1 wherein the chimeric co-stimulating receptor (CCR) comprises an extracellular domain and an intracellular domain joined by a transmembrane domain wherein the extracellular domain, expressed on the surface of the host immune cell, comprises an antigen binding domain having a binding affinity for the second antigen and the is intracellular domain delivers a costimulatory signal to the host immune cell but does not deliver an activation signal to the host immune cell.

13. The host immune cell of claim 12 wherein the antigen binding domain has a binding affinity for a second antigen selected from the group consisting of G-Protein Coupled Receptor 5D (GPRC5D), CD138, NY-ESO1, CD19 and SLAMF7/CS1.

14. The host immune cell of claim 13 wherein the antigen binding domain comprises a scFv that is specific for an epitope located in an extracellular domain of SLAMF7.

15. The host immune cell of claim 14 wherein the scFv comprises a VH domain having at least 90% of identity with the amino acid sequence as set forth in SEQ ID NO:15 and/or a VL domain having at least 90% of identity with the amino acid sequence as set forth in SEQ ID NO:16.

16. The host immune cell of claim 15 wherein the scFv comprises an amino acid sequence having at least 90% identity with the amino acid sequence as set forth in SEQ ID NO:17.

17. The host immune cell of claim 12 wherein the transmembrane domain comprises an amino acid sequence having at least 90% identity with the amino acid sequence as set forth in SEQ ID NO:12.

18. The host immune cell of claim 12 wherein the intracellular signalling domain of the CCR comprises one or more intracellular domain(s) of a costimulatory molecule selected from the group consisting of CD27, CD28, 4-1BB (CD137), OX40 (CD134), CD30, CD40, CD244 (2B4), ICOS, lymphocyte function-associated antigen-1 (LFA-1), CD2, CD7, LIGHT, NKG2C, B7-H3, and a ligand that specifically binds with CD83, CD8, CD4, b2c, CD80, CD86, DAP10, DAP12, MyD88, BTNL3, or NKG2D.

19. (canceled)

20. The host immune cell of claim 18 wherein the intracellular domain of the CCR comprises an amino acid sequence having at least 90% of identity with the amino acid sequence as set forth in SEQ ID NO:18.

21. The host immune cell of claim 12 wherein the CCR comprises an amino acid sequence having at least 90% identity with the amino acid sequence as set forth in SEQ ID NO:19.

22. (canceled)

23. (canceled)

24. (canceled)

25. (canceled)

26. (canceled)

27. (canceled)

28. A method of treating cancer in a subject in need thereof comprising administering to the subject a therapeutically effective amount of the host immune cell according to claim 1.

29. The method of claim 28 wherein the cancer is a CD38-positive hematological malignancy.

30. The method of claim 28, wherein the cancer is multiple myeloma.

31. (canceled)
